(19) United States
(12) Patent Application Publication
(10) Pub. No.: US 2025/0320258 A1
(43) Pub. Date: Oct. 16, 2025

(54) RNA RECOGNITION COMPLEX AND USES THEREOF

(71) Applicant: The Regents of the University of California, Oakland, CA (US)

(72) Inventors: Eugene Yeo, La Jolla, CA (US); Shengnan Xiang, La Jolla, CA (US); Frederick Tan, La Jolla, CA (US); Jonathan Schmok, La Jolla, CA (US)

(21) Appl. No.: 18/565,665

(22) PCT Filed: Jun. 1, 2022
(86) PCT No.: PCT/US2022/031780

§ 371 Date: Nov. 30, 2023

Related U.S. Application Data
(60) Provisional application No. 63/195,980, filed on Jun. 2, 2021.

(51) Int. Cl. C07K 14/165; A61K 38/16; A61K 48/00; C12N 9/22; C12N 15/113; C12N 15/85; C12Q 1/68

(52) U.S. Cl. CPC C07K 14/165; A61K 38/162; A61K 48/0008; C12N 9/222; C12N 15/113; C12N 15/85; C12Q 1/68; C12N 2770/20022

(57) ABSTRACT

Provided are RNA recognition complexes that include an RNA-targeting agent; and a coronavirus-derived protein. In some embodiments, the RNA recognition complex further includes a linker. In some embodiments, the RNA-targeting agent includes CRISPR/Cas9 components (e.g., a Cas9 protein, a Cas 13b protein, or a Cas 13d protein). Also provided herein are methods of upregulating gene expression of a target RNA that include delivering a RNA recognition complex into a cell, wherein the RNA recognition complex comprises a RNA-targeting agent, and a coronavirus-derived protein, and wherein the RNA recognition complex binds to the target RNA and upregulates gene expression of the target RNA in the cell.

### CROSS-REFERENCE TO RELATED APPLICATIONS

This application claims priority to U.S. Provisional Patent Application No. 63/195,980, filed on Jun. 2, 2021. The disclosure of the prior application is considered part of the disclosure of this application, and is incorporated herein by reference in its entirety.

### SEQUENCE LISTING

This application contains a Sequence Listing that has been submitted electronically as an ASCII text file named 156700352WO1_ST25. The ASCII text file, created on Jun. 1, 2022, is 1.46 kilobytes in size. The material in the ASCII text file is hereby incorporated by reference in its entirety.

### BACKGROUND

Recent transcriptome-wide and proteome-wide studies in viral protein-host protein interactions, viral protein and RNA interactions with host proteins, and viral RNA-host RNA interactions contribute to the understanding of host-virus interactions that are important to the SARS-CoV-2 virus life cycle and host response. However, the understanding of the RNA interactome of viral proteins remains limited.

It has been shown that the SARS-CoV-2 nucleocapsid protein interactome comprises many host RNA processing machinery proteins and stress granule proteins, suggesting a potential role in interfering with host RNA processing and driving stress granule formation. A majority of the viral proteins were found to associate with host RNA binding proteins (RBPs), suggesting a possibility that SARS-CoV-2 proteins interact with the host transcriptome to a greater degree than previously anticipated. However, a comprehensive interrogation of SARS-CoV-2 viral protein-host RNA interactions and how the virus hijacks host cellular machinery for its replication while it suppresses host gene expression is still lacking.

### SUMMARY

The present disclosure is based, at least in part, on RNA recognition complexes and methods of modulating gene expression of a target RNA using the RNA recognition complexes.

Provided herein are RNA recognition complexes comprising: (a) an RNA-targeting agent; and (b) a coronavirus-derived protein. In some embodiments, the RNA recognition complex further comprises a linker.

In some embodiments, the RNA-targeting agent comprises CRISPR/Cas9 components. In some embodiments, the RNA-targeting agent comprises an RNA-targeting Cas effector. In some embodiments, the RNA-targeting Cas effector comprises a Cas9 protein, a Cas13b protein, or a Cas13d protein. In some embodiments, the RNA-targeting Cas effector comprises a nulcease dead Cas9 (dCas9) protein. In some embodiments, the RNA-targeting Cas effector comprises a Cas13b protein. In some embodiments, the RNA-targeting Cas effector comprises a Cas13d protein.

In some embodiments, the RNA-targeting agent comprises a PUF protein. In some embodiments, the RNA-targeting agent comprises a pentatricopeptide repeat (PPR) protein.

In some embodiments, the RNA-targeting agent further comprises a single guide RNA (sgRNA), wherein the sgRNA is targeted to an individual gene of a cell. In some embodiments, the sgRNA is selected from a group consisting of SEQ ID NOs: 1-7.

In some embodiments, the coronavirus-derived protein comprises a SARS-CoV-2 protein. In some embodiments, the coronavirus-derived protein comprises a NSP1, a NSP2, a NSP3, a NSP6, a NSP12, a NSP14, a ORF3b, a ORF7b, or a ORF9c protein.

Also provided herein are methods of upregulating gene expression of a target RNA comprising: delivering a RNA recognition complex into a cell, wherein the RNA recognition complex comprises a RNA-targeting agent, and a coronavirus-derived protein, and wherein the RNA recognition complex binds to the target RNA and upregulates gene expression of the target RNA in the cell.

Also provided herein are methods of modulating gene expression of a target RNA comprising: delivering a RNA recognition complex into a cell, wherein the RNA recognition complex comprises a RNA-targeting agent, and a coronavirus-derived protein, and wherein the RNA recognition complex binds to the target RNA and modulates gene expression of the target RNA in the cell. In some embodiments, the method further comprises profiling the gene expression of the target RNA in the cell, wherein the gene expression is upregulated.

In some embodiments, the coronavirus-derived protein comprises a SARS-CoV-2 protein. In some embodiments, the coronavirus-derived protein comprises a NSP1, a NSP2, a NSP3, a NSP6, a NSP12, a NSP14, a ORF3b, a ORF7b, or a ORF9c protein.

In some embodiments, the method further comprises profiling the gene expression of the target RNA in the cell, wherein the gene expression is downregulated. In some embodiments, the coronavirus-derived protein comprises a NSP9 protein.

In some embodiments, the profiling comprises transcriptome analysis or gene expression analysis. In some embodiments, the profiling comprises enhanced cross-linking immunoprecipitation (eCLIP).

In some embodiments, the RNA-targeting agent comprises CRISPR/Cas9 components. In some embodiments, the RNA-targeting agent comprises an RNA-targeting Cas effector. In some embodiments, the RNA-targeting Cas effector comprises a Cas9 protein, a Cas13b protein, or a Cas13d protein. In some embodiments, the RNA-targeting Cas effector comprises a nulcease dead Cas9 (dCas9) protein. In some embodiments, the RNA-targeting Cas effector comprises a Cas13b protein. In some embodiments, the RNA-targeting Cas effector comprises a Cas13d protein.

In some embodiments, the RNA-targeting agent comprises a PUF protein. In some embodiments, the RNA-targeting agent comprises a pentatricopeptide repeat (PPR) protein.

In some embodiments, the RNA-targeting agent further comprises a single guide RNA (sgRNA), wherein the sgRNA is targeted to the target RNA in the cell. In some embodiments, the sgRNA is selected from a group consisting of SEQ ID NOs: 1-7.

Also provided herein are methods of treating a disease associated with reduced gene expression in a subject in need thereof, the method comprising: administering a RNA recognition complex to the subject, wherein the RNA recognition complex comprises a RNA-targeting agent, and a coronavirus-derived protein, and wherein the RNA recognition complex binds to the target RNA and upregulates gene expression of the target RNA in the cell, thereby treating the disease associated with reduced gene expression.

In some embodiments, the RNA-targeting agent comprises CRISPR/Cas9 components. In some embodiments, the RNA-targeting agent comprises an RNA-targeting Cas effector. In some embodiments, the RNA-targeting Cas effector comprises a Cas9 protein, a Cas13b protein, or a Cas13d protein. In some embodiments, the RNA-targeting Cas effector comprises a nulcease dead Cas9 (dCas9) protein. In some embodiments, the RNA-targeting Cas effector comprises a Cas13b protein. In some embodiments, the RNA-targeting Cas effector comprises a Cas13d protein.

In some embodiments, the RNA-targeting agent comprises a PUF protein. In some embodiments, the RNA-targeting agent comprises a pentatricopeptide repeat (PPR) protein.

In some embodiments, the RNA-targeting agent further comprises a single guide RNA (sgRNA), wherein the sgRNA is targeted to the target RNA in the cell. In some embodiments, the sgRNA is selected from a group consisting of SEQ ID NOs: 1-7.

In some embodiments, the coronavirus-derived protein comprises a SARS-CoV-2 protein. In some embodiments, the coronavirus-derived protein comprises a NSP1, a NSP2, a NSP3, a NSP6, a NSP12, a NSP14, a ORF3b, a ORF7b, or a ORF9c protein.

In some embodiments, the RNA-targeting agent comprises a sequence which is complementary to a target RNA sequence. In some embodiments, the RNA-targeting agent complementary sequence is at least 98% complementary to a target RNA sequence. In some embodiments, the RNA-targeting agent complementary sequence is at least 95% complementary to a target RNA sequence

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1a shows a schematic showing eCLIP performed on SARS-CoV-2 proteins in virus infected Vero E6 cells. Proteins in infected cells are UV crosslinked to bound transcripts, which are immunoprecipitated (IP) with antibodies that recognize NSP8, NSP12 and N proteins. Protein-RNA IP product and Input lysate are resolved by SDS-PAGE and membrane transferred, followed by band excision at the estimated protein size to 75 kDa above in both IP and Input lanes. Excised bands are subsequently purified, and library barcoded for Illumina sequencing. Sequenced reads are mapped to the hg19 human genome (GCF_000001405.13).

FIG. 1b is a bar plot showing number of all genes, number of all peaks number of coding genes and number of peaks mapping to coding genes from n=2 biologically independent replicates of NSP12, NSP8 and N eCLIP of SARS-CoV-2 infected cells.

FIG. 1c is a stacked bar plot showing TPM of reads mapped to the Vero E6 genome or SARS-CoV-2 genome in each of NSP12, NSP8 and N eCLIP.

FIG. 1d is a Venn diagram showing number of African Green Monkey (host) genes targeted by NSP8 and NSP12.

FIG. 1e shows eCLIP read density mapped to the SARS-CoV-2 genome on both the positive (top) and negative (bottom) sense strand.

FIG. if shows predicted secondary structure of the sequence from the NSP12 peak mapped to the C-terminal of NSP3.

FIG. 1g shows RNA-seq read density plot from SARS-CoV-2 infected A549-ACE2 cells mapping sequenced reads to the positive (top, blue) and negative (bottom, light blue) sense strand of SARS-CoV-2 genome.

FIG. 1h shows phylogenetic tree analysis of complete genomes of representative betacoronavirus from NCBI reference sequences and bat and pangolin coronavirus sequences from GISAID.

FIG. 1i shows predicted recombination events of SARS-CoV-2 from phylogenetic analysis, with line plot indicating significance (−log 10(P-value)) of predicted recombination breakpoints across the SARS-CoV-2 genome.

FIG. 2a shows a schematic showing SARS-CoV-2 proteins individually tagged and expressed in human lung epithelial cells BEAS-2B to assay with eCLIP.

FIG. 2b is a bar plot indicating number of all genes, number of all peaks, number of coding genes and number of coding peaks found to interact with each protein from n=2 biologically independent experiments. In addition to SARS-CoV-2 proteins, ENCODE eCLIP data for example human RNA-binding proteins (hRBPs) are included for comparison.

FIG. 2c is a Venn diagram showing the number of coding genes expressed at TPM>1.0 in Vero E6 and BEAS-2B cells as targeted by NSP12 with significant peaks (p<0.001, >4-fold enrichment).

FIG. 2d shows Circos plot mapping SARS-CoV-2 proteins to top five enriched Gene Ontology terms of host transcripts.

FIG. 2e shows example sequence logos generated from all IDR peak reads for each SARS-CoV-2 eCLIP, with p-value indicated above each logo.

FIG. 2f shows example genome browser tracks for NSP3, NSP12, N and NSP2 mapping to DYNCH1, TUSC3, CXCL5 and NAP1L4 respectively.

FIG. 3a shows stacked bar plot showing fraction of reproducible peaks (by IDR14) mapping to different regions of coding genes. 3ss, 3′ splice site; 3utr, 3′ untranslated region (UTR), 5ss, 5′ splice site; 5utr, 5′ UTR; CDS, coding sequence.

FIG. 3b shows example metagene profiles for NSP3, NSP12 and N. Mean of read density for each replicate data is shown as a solid line, with shaded regions indicating the 95% confidence interval.

FIG. 3c shows a schematic showing the Renilla-MS2 and Firefly dual luciferase reporter constructs, where individual SARS-CoV-2 proteins fused to MCP are recruited to the Renillia-MS2 mRNA.

FIGS. 3d and 3e show bar plots showing luciferase reporter activity ratios (FIG. 3d) and reporter RT-qPCR ratios (FIG. 3e) for the indicated coexpressed SARS-CoV-2 protein, known human regulators of RNA stability (CNOT7, BOLL) and negative control (FLAG peptide).

FIG. 3f shows bar plot showing the fold change of luciferase activity ratio and RT-qPCR 629 ratio.

FIGS. 3g and 3h show line plots show the fold enrichment of eCLIP read coverage at each position on rRNAs for NSP1 (FIG. 3g, blue) and ORF9c (FIG. 3h, blue), and the mean of 446 other RBPs deposited in the ENCODE consortium (grey; https://www.encodeproject.org/, accession code ENCSR456FVU) on 18S and 28S rRNAs (lightly shaded areas indicate 10-90% confidence intervals).

FIGS. 3i and 3j show quantitative flow cytometry reporter assay for targeted translation activation using RCas9-fused ORF9c.

FIG. 4a shows cumulative distribution plot (CDF) showing distribution of proteomics data from Bojkova et al2 of log 2(fold change) of host genes in SARS-CoV-2 infected vs. uninfected cells, for genes whose RNAs are not interacting with SARS-CoV-2 proteins, all eCLIP target genes (peak p<10-3, >8-fold enrichment), genes targeted by NSP12 (peak p<10-3, >8-fold enrichment), and genes targeted by NSP12 with highly significant peaks (peak p<10-7, >8-fold enrichment). P645 values are from KS test of the equality of log 2(fold change) of each subset of eCLIP target genes to the untargeted genes.

FIG. 4b shows top 10 Gene Ontology terms of NSP12 target genes.

FIG. 4c shows a map of NSP12 target genes (blue boxes connected by red edges to yellow box at center), clustered by top GO terms. Grey edges are human protein-protein interaction data from Mentha. Dark blue frames indicate genes used in subsequent validation.

FIG. 4d shows box plot showing quartiles of log 2(fold change) protein levels of NSP12 target genes from proteomics data grouped by the GO term classification. Mann-Whitney U-test p-values indicated above each box compares the log 2(fold change) of each subset of NSP12 target genes to all NSP12 target genes (red). Diamonds represent outliers, dots represent individual proteins.

FIG. 4e shows a schematic illustrating the hypothesis of NSP12 interacting with host mRNAs to upregulate the expression of target genes in mitochondrial and N-linked glycosylation processes.

FIG. 4f shows genome browser tracks of NSP12 eCLIP enriched RNA mapped to UGGT1, NDUFA4 and RPN1.

FIG. 4g shows western blots showing expression levels of UGGT1, NDUFA4 and RPN1, with β actin as loading control, from GFP or NSP12 transfected HEK293T cells. FIG. 4h shows immunofluorescence images (40×) of SARS-CoV-2 infected A549-ACE2 cells stained for SARS-CoV-2 NSP8 (red), endogenous genes (green), DNA content (blue).

FIG. 4i shows a bar plot showing mean relative fluorescence intensities of cells from FIG. 4h, dots represent segmented individual cells.

FIG. 5a shows a schematic illustrating NSP9 interacting with nuclear pore complex proteins NUP62, NUP214, NUP58, NUP88 and NUP541.

FIG. 5b shows a schematic showing the hypothesis of NSP9 inhibiting mRNA nucleocytoplasmic transport.

FIG. 5c shows genome browser tracks of NSP9 eCLIP target RNA mapped to IL-1α, IL-1β, ANXA2 and UPP1.

FIG. 5d shows a bar plot showing ratios of cytosolic to total fraction of mRNA levels measured by RT-qPCR, in wild type (WT) BEAS-2B cells, and BEAS-2B cells transduced to express NSP9 (*p<0.05, **p<0.0005, two-tailed multiple t-test with 672 pooled variance, n=2 biologically independent replicates).

FIG. 5e shows a bar plot showing mean concentration of IL-1α in culture media from WT and NSP9 expressing BEAS-2B cells, 48 h after induction by cytokines indicated on the x-axis.

FIG. 5f shows a bar plot showing mean concentration of IL-1α in culture media from WT and NSP9 expressing BEAS-2B cells, 48 h after induction by different levels of TNFα.

FIG. 5g shows a bar plot showing mean concentration of IL-1β in culture media from WT and NSP9 expressing BEAS-2B cells, 48 h after induction by 0 or 100 ng/ml TNFα (mean±s.e.m, n=3 biologically independent replicates, *p<0.05, **p<0.005, two-tailed test).

FIG. 6 shows a schematic illustrating the complex host-viral relationship. Flat-ended arrows indicate downregulation, pointed arrows indicate upregulation. Blue arrows are newly proposed interactions.

### DETAILED DESCRIPTION

This disclosure describes RNA recognition complexes and methods of modulating gene expression of a target RNA by delivering the RNA recognition complex into a cell.

Various non-limiting aspects of these methods are described herein, and can be used in any combination without limitation. Additional aspects of various components of methods for modulating gene expression are known in the art.

It must be noted that, as used in the specification and the appended claims, the singular forms “a,” “an” and “the” include plural referents unless the context clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, “biological sample” can refer to a sample generally including cells and/or other biological material. A biological sample can be obtained from non-mammalian organisms (e.g., a plants, an insect, an arachnid, a nematode), a fungi, an amphibian, or a fish (e.g., zebrafish). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a R-D-ribo configuration, α-LNA having an □-L-ribo configuration (a diastereomer of LNA), 2′-amino-LNA having a 2′-amino functionalization, and 2′-amino-□-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

Unless otherwise specified, a “nucleotide sequence encoding a protein” includes all nucleotide sequences that are degenerate versions of each other and thus encode the same amino acid sequence.

As used herein, the term “plurality” can refer to a state of having a plural (e.g., more than one) number of different types of things (e.g., a cell, a genomic sequence, a subject, a system, or a protein). In some embodiments, a plurality of nucleic acid sequences can be more than one nucleic acid sequence wherein each nucleic acid sequence is different from each other. In other embodiments, “plurality” can refer to a state of having a plural number of the same thing (e.g., a cell, a genomic sequence, a subject, a system, or a protein). In some embodiments, a plurality of nucleic acid sequences are identical to each other. In some embodiments, a plurality of cells are cellular clones (e.g., identical cells).

As used herein, the term “subject” is intended to include any mammal. In some embodiments, the subject is cat, a dog, a goat, a human, a non-human primate, a rodent (e.g., a mouse or a rat), a pig, or a sheep.

As used herein, the term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid encoding RNA-binding zinc finger domain.

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

### RNA Recognition Complex

As used herein, “RNA recognition complex” can refer to a system that can recognize specific mRNA transcripts and modulate protein expression. In some embodiments, an RNA recognition complex comprises an RNA-targeting agent and a coronavirus-derived protein. In some embodiments, the RNA-targeting agent can be fused or tethered to the coronavirus-derived protein.

As used herein, “RNA-targeting agent” can refer to an agent that can target and bind to a specific sequence in DNA or RNA. In some embodiments, an RNA-targeting agent comprises CRISPR/Cas9 components. As used herein, the term “CRISPR” refers to a technique of sequence specific genetic manipulation relying on the clustered regularly interspaced short palindromic repeats pathway, which unlike RNA interference regulates gene expression at a transcriptional level. In some embodiments, the RNA-targeting agent comprises a PUF protein. In some embodiments, the RNA-targeting agent comprises a pentatricopeptide repeat (PPR) protein. In some embodiments, the RNA-targeting agent comprises a protein that has an RNA binding domain.

As used here, in, “coronavirus-derived protein” can refer to a SARS-CoV-2 protein, and/or any variant thereof. In some embodiments, the coronavirus-derived protein includes a NSP1, a NSP2, a NSP3, a NSP6, a NSP12, a NSP14, a ORF3b, a ORF7b, or a ORF9c protein. In some embodiments, the coronavirus-derived protein includes a NSP9 protein.

In some embodiments, the RNA recognition complex further comprises a nuclear export signal and a coronavirus translation activation protein.

In some embodiments, an RNA recognition complex modulates protein expression in a temporal manner. In some embodiments, the RNA recognition complex can activate protein expression. In some embodiments, the RNA recognition complex can upregulate protein expression. In some embodiments, the RNA recognition complex can downregulate protein expression.

### RNA-Targeting Agents

### CRISPR Cas Systems

In some embodiments, an RNA-targeting agent is an RNA-guided target RNA-binding fusion protein. RNA-guided target RNA-binding fusion proteins comprise at least one RNA-binding polypeptide which corresponds to a gRNA which guides the RNA-binding polypeptide to target RNA. RNA-guided target RNA-binding fusion proteins include without limitation, RNA-binding polypeptides which are CRISPR/Cas-based RNA-binding polypeptides or portions thereof.

In some embodiments, the RNA-targeting agent comprises an RNA-targeting Cas effector. As used herein, a “Cas effector” or “CRISPR-associated protein” can refer to an enzyme or protein that uses CRISPR sequences as a guide to recognize and cleave specific nucleic acid strands that are complementary to the CRISPR sequence. An RNA-targeting Cas effector can associate with a CRISPR RNA sequence to bind to, and alter DNA or RNA target sequences. In some embodiments, an RNA-targeting Cas effector can be a Cas9 endonuclease that makes a double-stranded break in a target DNA sequence. In some embodiments, an RNA-targeting Cas effector can be a Cas12a nuclease that also makes a double-stranded break in a target DNA sequence. In some embodiments, an RNA-targeting Cas effector can be a Cas13 nuclease which targets RNA. In some embodiments, the RNA-targeting Cas effector comprises a Cas9 protein, a Cas13b protein, or a Cas13d protein. In some embodiments, the RNA-targeting Cas effector comprises a nuclease dead Cas9 (dCas9) protein. In some embodiments, the RNA-targeting Cas effector comprises a Cas13b protein. In some embodiments, the RNA-targeting Cas effector comprises a Cas13d protein.

In some embodiments, the RNA-targeting agent further comprises a single guide RNA (sgRNA), wherein the sgRNA is targeted to an individual gene of a cell. The term “single guide RNA” or “sgRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the single guide RNA can recognize a target RNA, for example, by hybridizing to the target RNA. In some embodiments, the single guide RNA comprises a sequence that is complementary to the target RNA. In some embodiments, the sgRNA can include one or more modified nucleotides. In some embodiments, the sgRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt). In some embodiments, the sgRNA can include a sequence from SEQ ID NOs: 1-7 (Table 1).

TABLE 1

| | Sequence 5′ → 3′ | SEQ ID |
|---|---|---|
| gRNA Name | (mRNA sequence) | NO |
| g.5utr1 | ttttgacctccatagaagac | 1 |
| g.5utr2 | ggacggacgccagcgctaag | 2 |
| g.5utr3 | atccccgggtaccggtcgcc | 3 |
| g.cds1 | cggtcgccaccatggtgagc | 4 |
| g.cds2 | gaccacctteggctacggcc | 5 |
| g.3utr1 | attcttacgctgagtacttc | 6 |
| g.3utr2 | catggcattccacttatcac | 7 |

In some embodiments, a single guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as cancers, neurodegeneration, cutaneous conditions, endocrine conditions, intestinal diseases, infectious conditions, neurological conditions, liver diseases, heart disorders, or autoimmune diseases. In some embodiments, a target RNA can be a therapeutic target for conditions such as cancers, neurodegeneration, cutaneous conditions, endocrine conditions, intestinal diseases, infectious conditions, neurological conditions, liver diseases, heart disorders, or autoimmune diseases. In some embodiments, the sgRNA can be driven by a promoter. In some embodiments, the promoter can be a U6 polymerase III promoter.

### PUF Proteins

In some embodiments, a RNA-targeting agent is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family). This type of RNA-binding polypeptide can be used in place of a gRNA-guided RNA binding protein such as CRISPR/Cas. The unique RNA recognition mode of PUF proteins (named for Drosophila Pumilio and C. elegans fem-3 binding factor) that are involved in mediating mRNA stability and translation are well known in the art. The PUF domain of human Pumiliol, also known in the art, binds tightly to cognate RNA sequences and its specificity can be modified. It contains eight PUF repeats that recognize eight consecutive RNA bases with each repeat recognizing a single base. Since two amino acid side chains in each repeat recognize the Watson-Crick edge of the corresponding base and determine the specificity of that repeat, a PUF domain can be designed to specifically bind most 8-nt RNA. Wang et al., Nat Methods. 2009; 6(11): 825-830. See WO2012/068627, which is incorporated by reference herein in its entirety, for additional disclosure regarding PUF proteins.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD (Pumilio homology domain, a member of the PUF family), which has been widely used in native and modified form for targeting RNA, has been engineered to yield a set of four canonical protein modules, each of which targets one RNA base. These modules (i.e., Pumby, for Pumilio-based assembly) can be concatenated in chains of varying composition and length, to bind desired target RNAs. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593, which is incorporated by reference herein in its entirety, for additional disclosure regarding PUMBY proteins.

In some embodiments of the compositions of the disclosure, the RNA-targeting agent comprises a Pumilio and FBF (PUF) protein. In some embodiments, the RNA-targeting agent comprises a Pumilio-based assembly (PUMBY) protein.

### PPR Proteins

In some embodiments of the compositions of the disclosure, at least one of the RNA-binding proteins or RNA-binding portions thereof is a PPR protein (proteins with pentatricopeptide repeat (PPR) motifs derived from plants). PPR proteins are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. See WO 2013/058404, which is incorporated herein by reference in its entirety for additional disclosure regarding PPR proteins.

### Coronavirus-Derived Protein

Coronaviruses contain a positive-sense, single-stranded RNA genome, and the viral genome consists of more than 29,000 bases and encodes 29 proteins. SARS-CoV-2 has four structural proteins: the E and M proteins, which form the viral envelope; the N protein, which binds to the virus's RNA genome; and the S protein, which binds to human receptors. As used herein, “coronavirus-derived protein” can refer to a protein that is encoded from the coronavirus viral genome. In some embodiments, the coronavirus-derived protein can be a non-structural protein (NSP). In some embodiments, the non-structural protein can comprise a NSP1, a NSP2, a NSP3, a NSP4, a NSP5, a NSP6, a NSP7, a NSP8, a NSP9, a NSP10, a NSP12, a NSP13, a NSP14, a NSP15, or a NSP16 protein. In some embodiments, the coronavirus-derived protein can be an accessory protein. In some embodiments, the accessory protein can comprise a ORF3a, a ORF6, a ORF7a, a ORF7b, a ORF8, or a ORF10 protein. In some embodiments, the coronavirus-derived protein can be a structural protein. In some embodiments, the structural protein can comprise a spike (S) protein, a nucleocapsid (N) protein, a membrane (M) protein, or an envelope (E) protein. In some embodiments, the coronavirus-derived protein comprises a NSP1, a NSP2, a NSP3, a NSP6, a NSP12, a NSP14, a ORF3b, a ORF7b, or a ORF9c protein. In some embodiments, the coronavirus-derived protein comprises a NSP9 protein.

### Linker

In some embodiments, the RNA recognition complex disclosed herein comprises a linker between the RNA-targeting agent and the coronavirus-derived protein. In some embodiments, the linkers or linker motifs can be any flexible peptides that connect two protein domains or motifs without interfering with their functions. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker. See WO2017/192434, WO2019/089817, and WO2019/241483, each of which are herein incorporated in its entirety, for more disclosure regarding using linkers.

### Nucleic Acids

Provided herein are the nucleic acid sequences encoding the RNA recognition complexes disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids can include alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein.

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In some embodiments, the vector further comprises a nucleic acid corresponding to an RNA recognition complex of the disclosure. In some embodiments, the RNA recognition complex comprises a RNA targeting agent and a coronavirus-derived protein.

In some embodiments of the compositions and methods of the disclosure, a first vector comprises a guide RNA of the disclosure and a second vector comprises a RNA recognition complex of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the RNA recognition complex comprises a RNA targeting agent and a coronavirus-derived protein. In some embodiments, the first vector and the second vector are identical. In some embodiments, the first vector and the second vector are not identical.

In some embodiments of the compositions and methods of the disclosure, a vector of the disclosure is a viral vector. In some embodiments, the viral vector includes a sequence isolated or derived from a retrovirus. In some embodiments, the viral vector includes a sequence isolated or derived from a lentivirus. In some embodiments, the viral vector includes a sequence isolated or derived from an adenovirus. In some embodiments, the viral vector includes a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector includes a sequence isolated or derived from an adeno-associated virus (AAV). In some embodiments, the viral vector includes an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, AAV12, AAV.rh32/33, AAV.rh43, AAV.rh64R1, and any combinations or equivalents thereof. In some embodiments, the viral vector is replication incompetent. In some embodiments, the viral vector is isolated or recombinant (rAAV). In some embodiments, the viral vector is self-complementary (scAAV). In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb.

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nP2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVsM) vector, a modified sooty mangabey simian immunodeficiency virus (SIVsM) vector, a African green monkey simian immunodeficiency virus (SIVAGm) vector, a modified African green monkey simian immunodeficiency virus (SIVAGm) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Pharmaceutical Compositions

The methods described herein can include the administration of pharmaceutical compositions and formulations including vectors delivering an RNA recognition complex including an RNA-targeting agent and a coronavirus-derived protein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The RNA recognition complex can be administered alone or as a component of a pharmaceutical formulation (composition). The compounds may be formulated for administration, in any convenient way for use in human or veterinary medicine. The compositions may conveniently be presented in unit dosage form and may be prepared by any methods well known in the art of pharmacy. The amount of active ingredient which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect.

Pharmaceutical compositions described herein can be prepared according to any method known to the art for the manufacture of pharmaceuticals. Such compositions can contain, for example, preserving agents. A composition can be admixtured with nontoxic pharmaceutically acceptable excipients which are suitable for manufacture. Compositions may comprise one or more diluents, emulsifiers, preservatives, buffers, excipients, etc. and may be provided in such forms as liquids, powders, emulsions, lyophilized powders, controlled release formulations, on patches, in implants, etc. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences as described herein. As an example of an injectable oil vehicle, see Minto (1997) J. Pharmacol. Exp. Ther. 281:93-102.

Pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

In some embodiments, the pharmaceutical compositions can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compositions can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860. Compositions disclosed herein can be administered for prophylactic and/or therapeutic treatments. In some embodiments, for therapeutic applications, compositions are administered to a subject who is infected or at risk of infection with SARS-CoV2, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease the number of lung cells infected with SARS-CoV2.

The inhibitory nucleic acids used to practice the methods described herein, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect, or plant cell expression systems.

### Modulating Gene Expression of a Target RNA

In some embodiments, a method of upregulating gene expression of a target RNA can include delivering a RNA recognition complex into a cell, wherein the RNA recognition complex comprises a RNA-targeting agent, and a coronavirus-derived protein, and wherein the RNA recognition complex binds to the target RNA and upregulates gene expression of the target RNA in the cell.

In some embodiments, a method of modulating gene expression of a target RNA can include delivering a RNA recognition complex into a cell, wherein the RNA recognition complex comprises a RNA-targeting agent, and a coronavirus-derived protein, and wherein the RNA recognition complex binds to the target RNA and modulates gene expression of the target RNA in the cell.

In some embodiments, the RNA recognition complex is present in a delivery system. In some embodiments, the delivery system comprises a delivery vehicle selected from the group consisting of an adeno-associated virus, a nanoparticle, and a liposome.

In some embodiments, the RNA recognition complex can be introduced into any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. In some embodiments, the RNA recognition complex can be delivered into the cytoplasm of a cell. In some embodiments, the RNA recognition complex can be delivered into the cell by chemical transfection, non-chemical transfection, particle-based transfection, or viral transfection. In some embodiments, the RNA recognition complex can be delivered with a transfection reagent. In some embodiments, the transfection reagent can be lipofectamine. In some embodiments, the transfection reagent can be FuGENE transfection reagent.

In some embodiments, the method further includes profiling the gene expression of the target RNA in the cell, wherein the gene expression is upregulated. In some embodiments, a target RNA, through an RNA-targeting agent's association with a coronavirus-derived protein, drives upregulation of the target RNA within a cell. In some embodiments, the coronavirus-derived protein comprises a NSP1, a NSP2, a NSP3, a NSP6, a NSP12, a NSP14, a ORF3b, a ORF7b, or a ORF9c protein.

In some embodiments, the method further includes profiling the gene expression of the target RNA in the cell, wherein the gene expression is downregulated. In some embodiments, a target RNA, through an RNA-targeting agent's association with a coronavirus-derived protein, drives downregulation of the target RNA within a cell. In some embodiments, the coronavirus-derived protein comprises a NSP9 protein.

As used herein, “profiling” can refer to the measurement of activity (e.g., expression) of one or more genes, to create a global picture of cellular function. In some embodiments, profiling includes sequencing of a nucleic acid (e.g., DNA or RNA), wherein the gene expression profile includes information of active translation at a point in time. In some embodiments, the profiling comprises transcriptome analysis or gene expression analysis. In some embodiments, the profiling comprises enhanced cross-linking immunoprecipitation (eCLIP). As used herein, “enhanced crosslinking and immunoprecipitation (eCLIP)” refers to a method to profile RNAs bound by an RNA binding protein of interest. In some embodiments, eCLIP can be modified and used to profile RNAs bound by specific ribosomal subunit proteins. In some embodiments, enhanced crosslinking and immunoprecipitation (eCLIP) recovers protein-coding mRNAs (with a particular enrichment for coding sequence regions).

As used herein, “immunoprecipitation” is the technique of precipitating a protein antigen out of solution using an antibody that specifically bind to that particular protein. In some embodiments, the solution containing the protein antigen is in the form of a crude lysate of an animal tissue. Immunoprecipitation can be used to isolate and concentrate a particular protein from a sample containing many different proteins. Also, this technique requires that the antibody by coupled to a solid substrate (e.g., immunoprecipitation beads) while performing the procedure. Existing crosslinking and immunoprecipitation (CLIP) methods also identify RNA nucleotides that bind proteins of interest, but typically deliver regions up to hundreds of nucleotides in length that are the approximate binding sites of the given protein. Enhanced crosslinking and immunoprecipitation (eCLIP) is a method to profile RNAs bound by an RNA binding protein of interest.

### Methods of Treating

In some embodiments, a method of treating a disease of reduced gene expression in a subject in need thereof can include administering a RNA recognition complex to the subject, wherein the RNA recognition complex comprises a RNA-targeting agent, and a coronavirus-derived protein, and wherein the RNA recognition complex binds to the target RNA and upregulates gene expression of the target RNA in the cell.

### EXAMPLES

The disclosure is further described in the following examples, which do not limit the scope of the disclosure described in the claims.

### Example 1—eCLIP Elucidates SARS-CoV-2 Protein-RNA Interactions in Virus Infected Cells

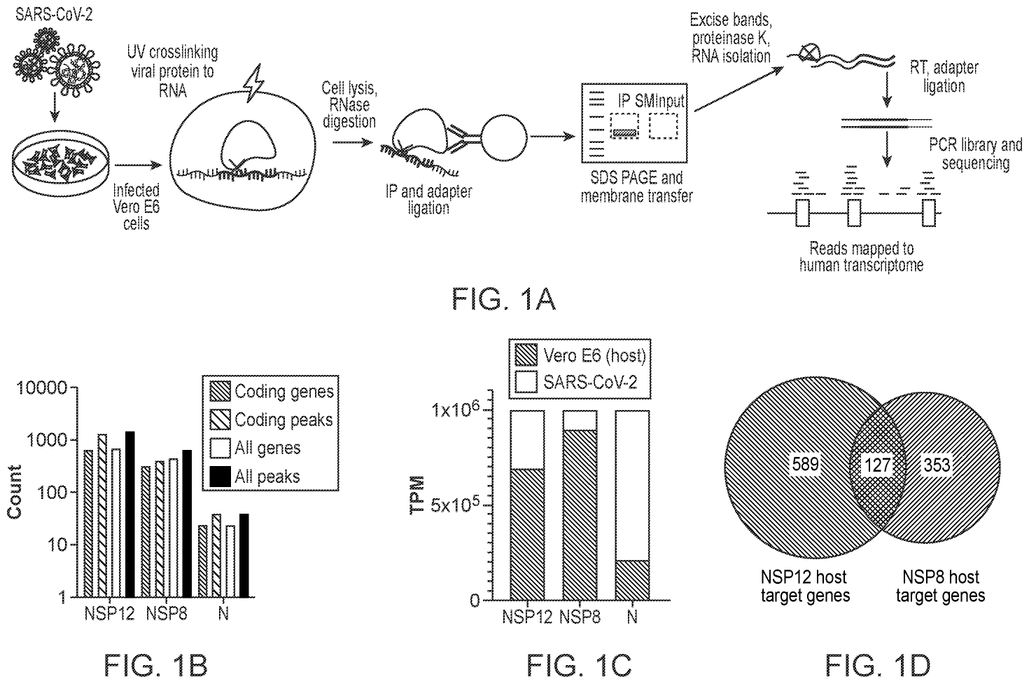

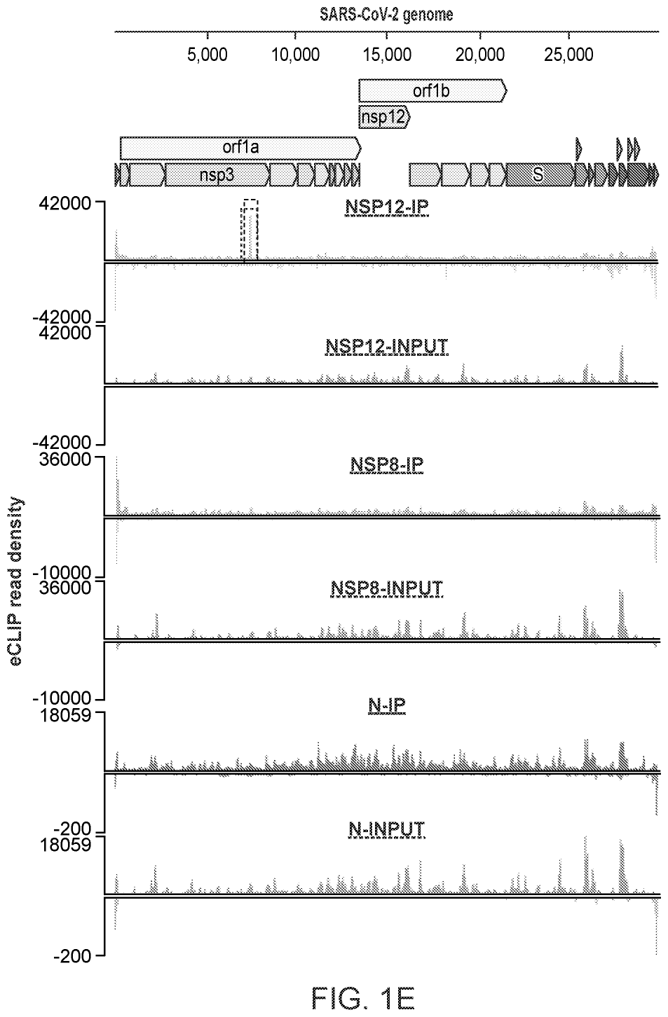

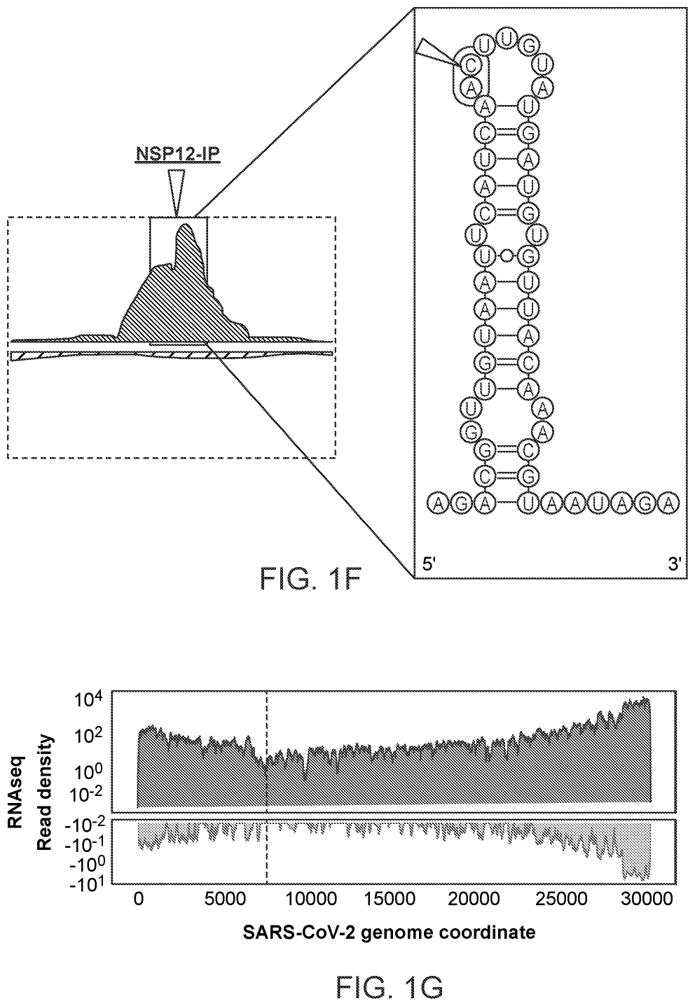

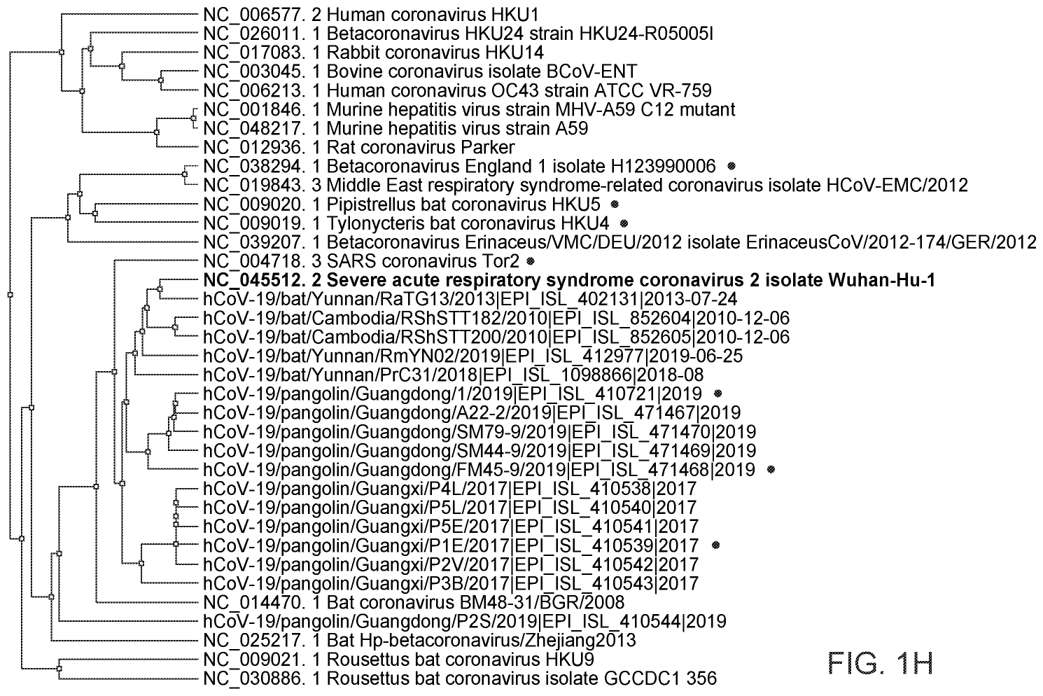

To investigate the RNA interactome of SARS-CoV-2 proteins, eCLIP was performed on SARS-CoV-2 infected African Green Monkey kidney (Vero E6) cells (FIG. 1a). Cells were infected at a multiplicity of infection (MOI) of 0.01 for 48 hours before UV irradiation of cells that covalently crosslink interacting proteins to RNAs. This was followed by immunoprecipitation of the NSP8, NSP12 (also known as the RNA dependent RNA polymerase) and N (nucleocapsid) proteins using protein-specific antibodies to isolate the bound RNA. The RNA-bound proteins were resolved via SDS-PAGE and transferred to nitrocellulose membranes such that only the region spanning the expected protein size and 75 kDa larger were excised and purified in subsequent steps. The same size region of a non-immunoprecipitated input whole cell lysate was included as size-matched input to identify enriched sequences. RNA was converted to libraries and sequenced to an average depth of ˜25 million reads, and mapped to the SARS-CoV-2 viral genome and African Green Monkey genome to determine SARS-CoV-2 protein RNA interactions. Targeted transcripts were determined by having one or more peaks that meet the stringent IDR (irreproducible discovery rate) threshold of overlapping peaks between two replicates for every protein, and satisfy statistical cutoffs of p<0.001, and more than 8-fold enrichment in the immunoprecipitated sample (IP) over the size-matched input sample.

It was found that NSP8, NSP12 and N interact with 457, 703 and 24 genes with 658, 1457 and 39 significant peaks, respectively (FIG. 1b). The number of RNA reads in Transcripts Per Kilobase Million (TPM) from both NSP8 and NSP12 immunoprecipitation (IP) samples were mapped more frequently to host transcripts than viral RNA (FIG. 1c). In contrast, a majority of N immunoprecipitated RNA reads were mapped to viral RNA, consistent with its role in enclosing the viral genome during virion assembly. All three proteins bound to viral RNA with peaks that were highly statistically significant (p-values <10⁻⁴⁰⁰), although the large number of peaks (2137) that map to the host genes suggests a potential role in their regulation (FIG. 1d).

The eCLIP results provide the first viral RNA genome map of interactions with NSP8, NSP12 and N proteins. We observed strong NSP8 and NSP12 eCLIP peaks at the 5′ untranslated region (UTR) and 3′ UTR of both positive and negative strand viral transcripts (FIG. 1e). This is consistent with the role of replicase proteins NSP8 and NSP12 in viral genome replication. Furthermore, NSP12 enrichment was seen on the negative strand at all transcription-regulatory sequences (TRSs) of the viral genome, implying that it may play a role in the transcription of subgenomic RNAs, which results in the expression of accessory protein products. However, no enrichment of eCLIP reads were observed on the positive sense strand for TRSs in eCLIP of NSP12, and NSP8 and N eCLIP reads were not enriched at the TRSs of either strand. In fact, very few distinct peaks were identified from the eCLIP results of N, as the eCLIP reads were nonspecifically distributed across the genome, indistinguishable to the input sample (FIG. 1e). This is consistent with the nucleocapsid encapsulating the entire viral genome in the packaged viral particles.

Unexpectedly, a distinct NSP12 eCLIP peak at the region around position 7450-7550 in the positive sense strand was observed, near the 3′ end of the gene encoding for NSP3. Upon closer inspection, the eCLIP read density showed a sharp drop in reads at position 7481 on both strands, which may correspond to reverse transcription termination during eCLIP library preparation at a UV crosslinking site (FIG. 1f). Within this region, the sequence at position 7470-7510 forms a stable hairpin from RNA secondary structure prediction. The high read density in the hairpin region suggests a potential stalling of NSP12 polymerase elongation, which may result in aborted transcripts. In support of this hypothesis, RNA-seq of A549-ACE2 cells infected with SARS-CoV-2 was performed and a steep decrease in transcript read density at the site of NSP12 eCLIP peak was observed (FIG. 1g). Aborted transcripts were also confirmed in a direct RNA-sequencing study using the Oxford Nanopore18. Furthermore, some of these aborted transcripts join up with the downstream sequences, forming deletion products.

Polymerase stalling may play a role in generating genetic diversity of viruses via recombination, which has been shown to contribute to the evolution of SARS-CoV-2. To determine the likelihood of recombination across the viral genome, a multiple sequence alignment and phylogenetic analysis of the reference sequences of the complete genomes of betacoronaviruses from NCBI and the complete genomes of bat and pangolin coronaviruses from GISAID was performed (FIG. 1h). The multiple sequence analysis shows the peak region sequence to be highly conserved among the analyzed betacoronavirus sequences. The hairpin structure also appears conserved among bat and pangolin sequences. Recombination breakpoints are predicted from this sequence alignment, using a pairwise scanning approach that identifies regions with greater similarity among phylogenetically distant sequences. The prediction found a likely breakpoint ˜250 nt downstream of the peak in region 7450-7550. This breakpoint was predicted to be a recombination event between SARS-CoV-2 and the Tylonycteris bat coronavirus HKU4 (FIG. 1i). While there are several other breakpoints that did not coincide with NSP12 eCLIP peaks, the presence of the NSP12 eCLIP peak in the 7470-7510 region proximal to a potential recombination site suggests a possible contribution to recombination in ancestral sequences of SARS-CoV-2. In addition to a high degree of sequence conservation, the RNA secondary structure appears conserved in the region containing the 7470-7510 peak among the closely related pangolin and bat betacoronaviruses, suggesting a potential function associated with NSP12 binding to this region that may be important for virus replication.

Taken together, the first eCLIP data showing the interaction of SARS-CoV-2 proteins NSP8, NSP12 and N bound to the viral genome is presented. These findings suggest that NSP12 may be involved in transcription stalling and contribute to viral genetic diversity via recombination. The large number of host RNAs bound by NSP12 prompted a systematic investigation of SARS-CoV-2 protein-host RNA interactions.

### Example 2—SARS-CoV-2 Proteins Interact with One Third of the Transcriptome in Lung Epithelial Cells

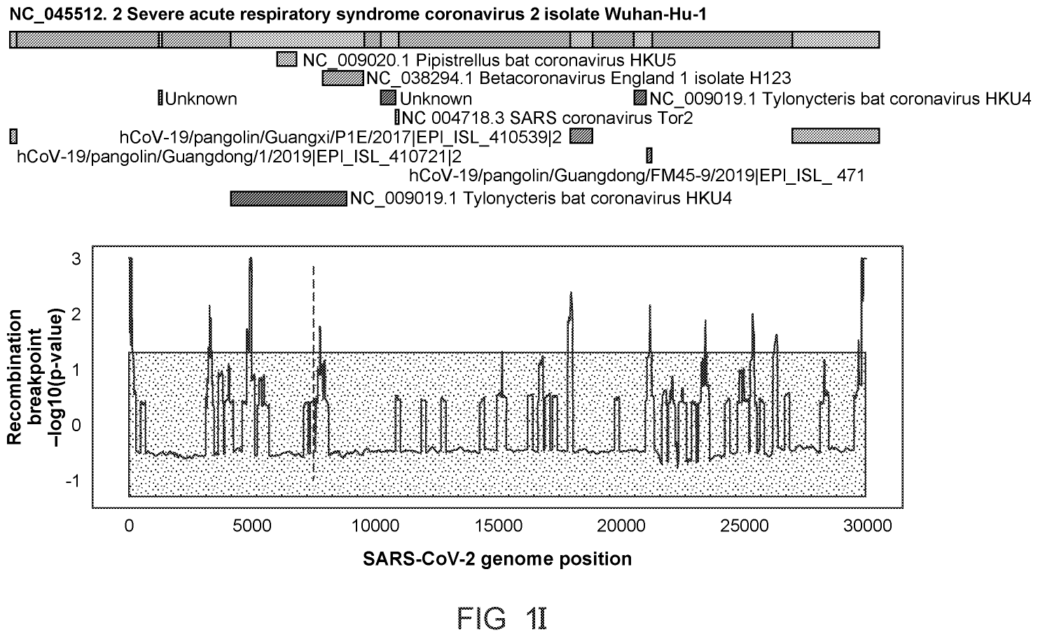

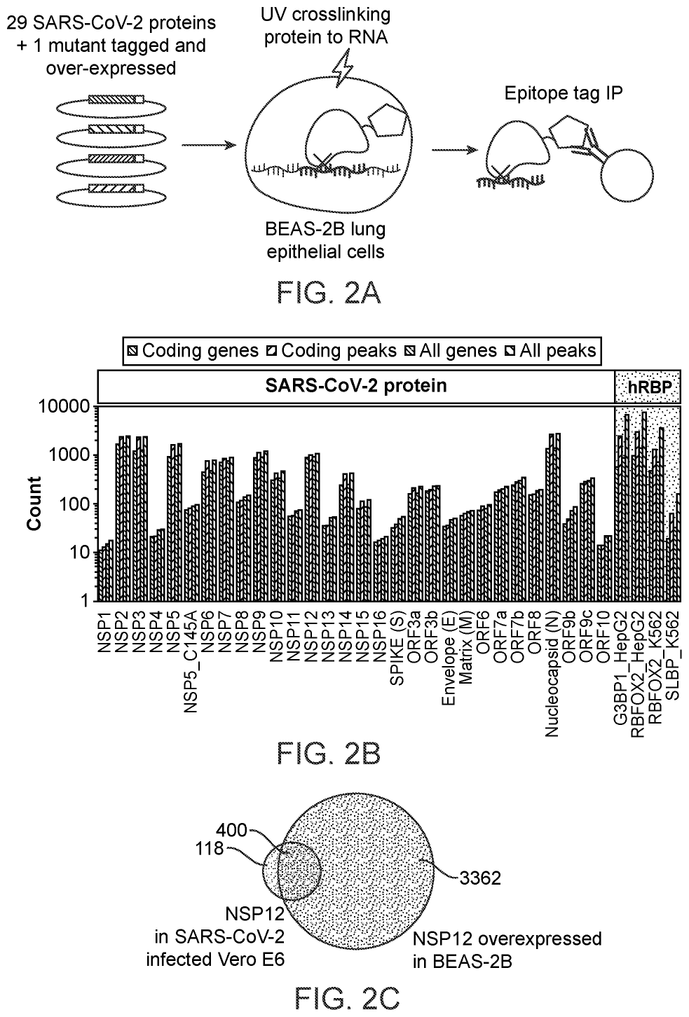

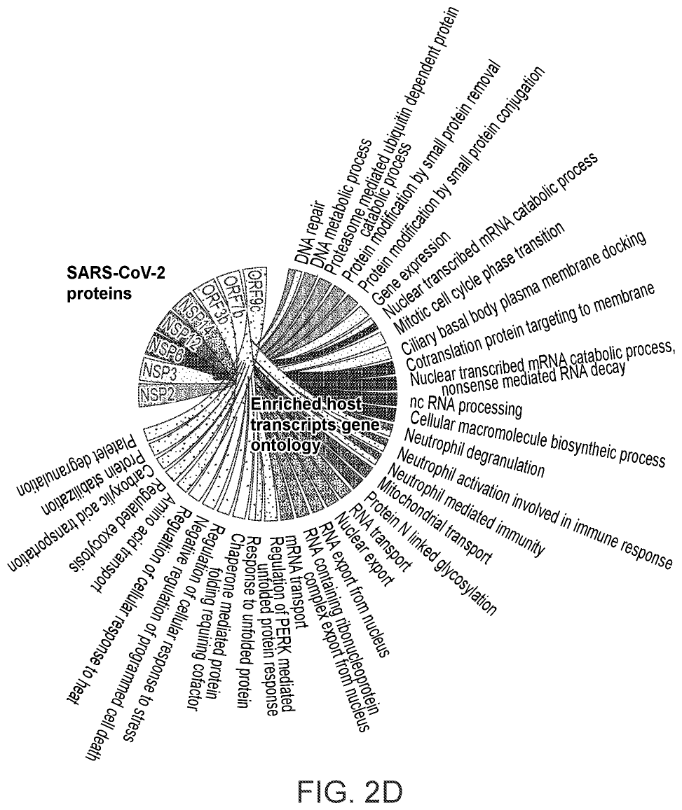

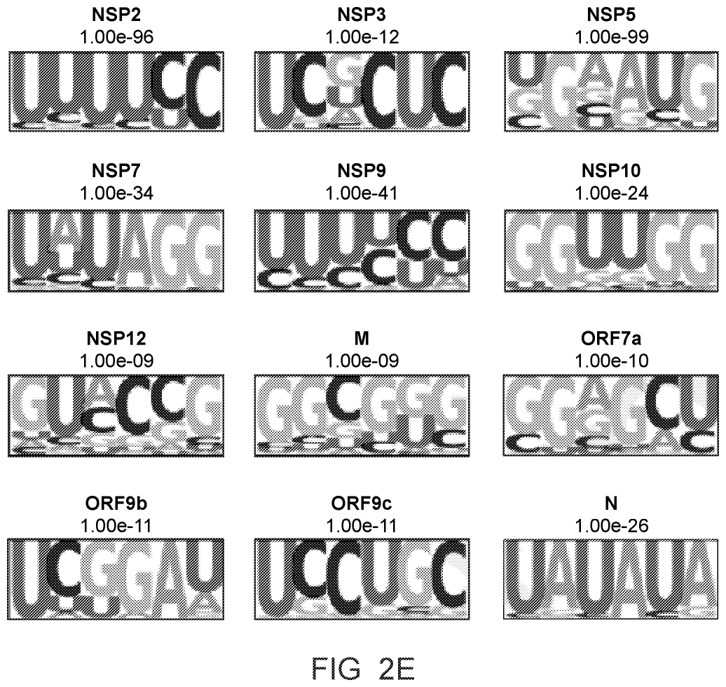

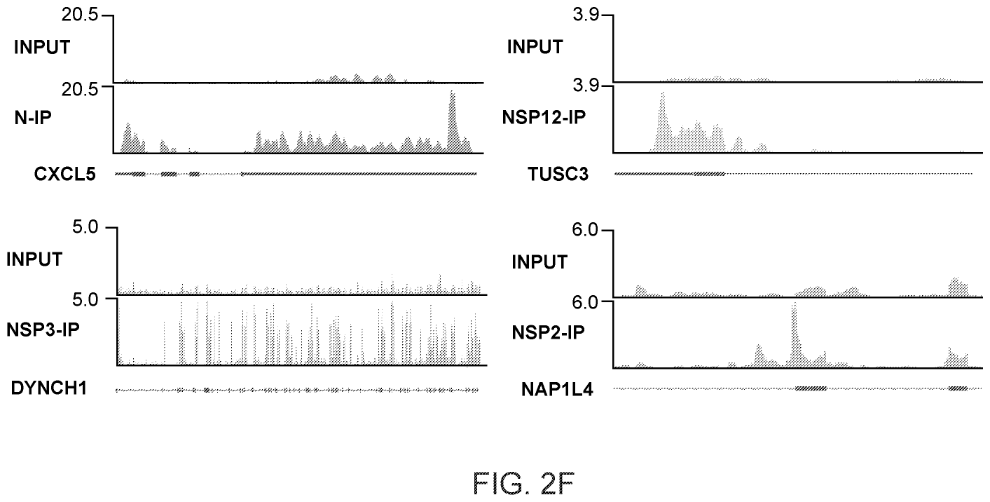

To investigate whether SARS-CoV-2 proteins directly interact with the human host transcriptome, eCLIP was performed on the 29 proteins encoded in the SARS-CoV-2 genome and one mutant (FIG. 2a). Due to the lack of antibodies specific for most of the viral proteins, the individual proteins were overexpressed in a lung epithelial cell line BEAS-2B, which is an immortalized primary bronchial cell line representative of normal lung physiology. Each protein was either fused with a 2×Strep tag and expressed stably via lentiviral transduction or fused with a 3×FLAG tag and expressed transiently via transfection. Following UV crosslinking, the tagged proteins were immunoprecipitated using anti-FLAG or anti-Strep antibodies.

From the SARS-CoV-2 proteome-wide eCLIP results, SARS-CoV-2 proteins interacted with RNA represented by 4,821 coding genes, which is about a third of the transcriptome of BEAS-2B cells. Nucleocapsid and non-structural proteins NSP2, NSP3, NSP5, NSP9 and NSP12 were found to target the greatest number of unique genes at 1339, 1647, 1199, 902, 863, and 865, respectively (FIG. 2b). The large number of genes targeted by the viral proteins is consistent with the non-structural proteins from the replicase (ORF1ab) having a high affinity for its own RNA, though their potential for widespread interaction with host RNA has not been shown previously. The widespread interaction of Nucleocapsid with host RNAs when expressed in isolation is consistent with its capacity for nonspecific RNA binding, whereas its targeting the virus genome during RNA assembly occurs via interaction with the M protein. For comparison, the extensively studied splicing factor RBFOX2 binds to 958 genes in HepG2 cells and 471 genes in K562 cells, the stress granule assembly factor G3BP1 binds to 561 genes in HepG2 cells, and the histone RNA hairpin-binding protein SLBP binds to 19 genes in K562 (FIG. 2b). This suggests that viral proteins have the same capacity for interacting with RNA as endogenous human RBPs. Most of the target genes (400/518) in the NSP12 eCLIP in virus infected Vero E6 cells are represented in the eCLIP assay from exogenous expression in the BEAS-2B cells (FIG. 2c). Only transcripts that are expressed at a TPM of >1.0 in both cell lines are used in this comparison, and target genes are considered if bound by one or more peaks that satisfy statistical cutoffs of −log 10(p-value) >3, and more than 4-fold enrichment over size-matched input. This suggests that NSP12 bound genes are similar in the context of the virus infected cells and in the context of NSP12 expressed in isolation.

Distinct processes related to viral replication and host response are targeted by the viral proteins as shown by gene ontology (GO) analysis (FIG. 2d). Many of the enriched GO terms are related to nucleic acid and protein synthesis, modification and transport, which is consistent with the primary objective of the virus hijacking host resources for its own biosynthesis and replication. A few stress response processes are enriched, including response to heat, as targeted by ORF7b. Immune response processes are also enriched, including neutrophil mediated immunity targeted by NSP12 and platelet degranulation targeted by ORF9c. This supports the choice of lung epithelial cells as a model system that expresses the relevant cytokines for recruiting immune cells. In addition to immune response, ciliary basal body plasma membrane docking genes are enriched, which may be related to ciliated lung cells as the site of viral entry. While the enriched GO terms are highly relevant to viral and host response processes, further analysis of binding patterns is needed to determine if there are any functional implications of viral proteins interacting with these genes.

To determine if there are sequence features that the viral proteins recognize, sequence logos were generated from 6-mers of the bound RNA reads. While some of the proteins display strong sequence preferences (FIG. 2d) other proteins appear to bind more non-specifically. Some motifs resemble enrichments observed for human RBPs, where M, ORF7a and NSP10 appear to favor G-rich or GU rich motifs, and NSP5 has a motif (GNAUG). Other motifs may result from regional binding preferences (FIG. 2e), as NSP2 and NSP9 have a strong preference for UC-rich polypyrimidine motifs (p values of 10-96 and 10-41 respectively), which may be a result of their binding to polypyrimidine tracts in intronic regions, whereas N has an AU-rich motif likely because it preferentially binds to 3′ UTR which contain AU-rich elements. NSP3, a large multifunctional protein, appears to coat entire transcripts and may not have a meaningful sequence motif. NSP12 primarily binds in the 5′ UTR, and a weakly enriched GUCCCG motif that resembles terminal oligopyrimidine (TOP) motifs hints at a possible role in translation regulation.

The systematic interrogation of SARS-CoV-2 protein-host RNA interactions demonstrates that a majority of SARS-CoV-2 viral proteins are RNA binding proteins that target a third of the human transcriptome. The analysis implies that these viral proteins may be involved in perturbing many essential cellular processes of the host. In addition, SARS-CoV-2 protein specific antibodies enabled confirming the large number of interactions between viral proteins NSP12 and NSP8 and host RNAs in the context of the intact and live virus. As eCLIP in virus infected cells are limited by IP-grade antibodies, focus was placed on the data obtained from the exogenous expression of individual proteins in BEAS-2B cells for systematic analysis of potential functional implications.

### Example 3—Select SARS-CoV-2 Proteins Upregulate Protein Expression of Target Transcripts

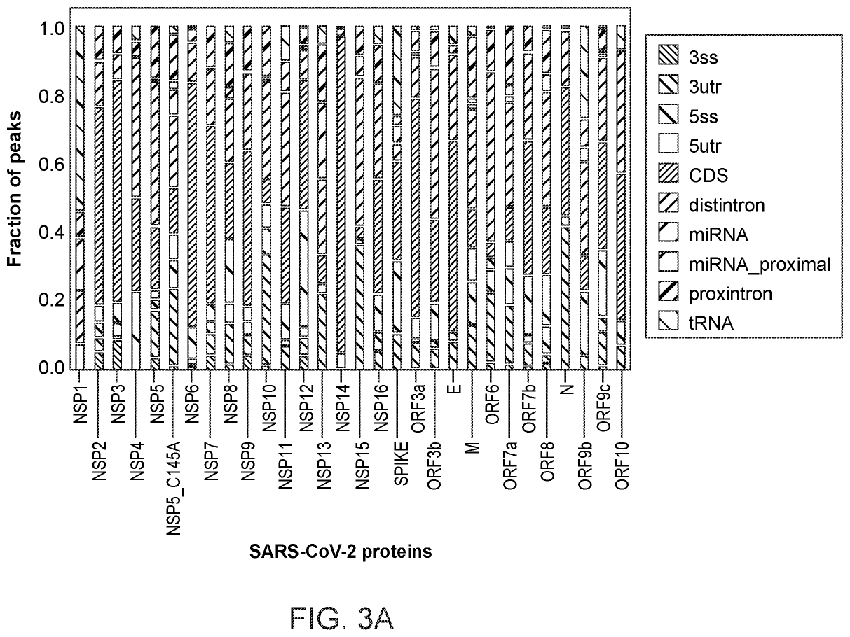

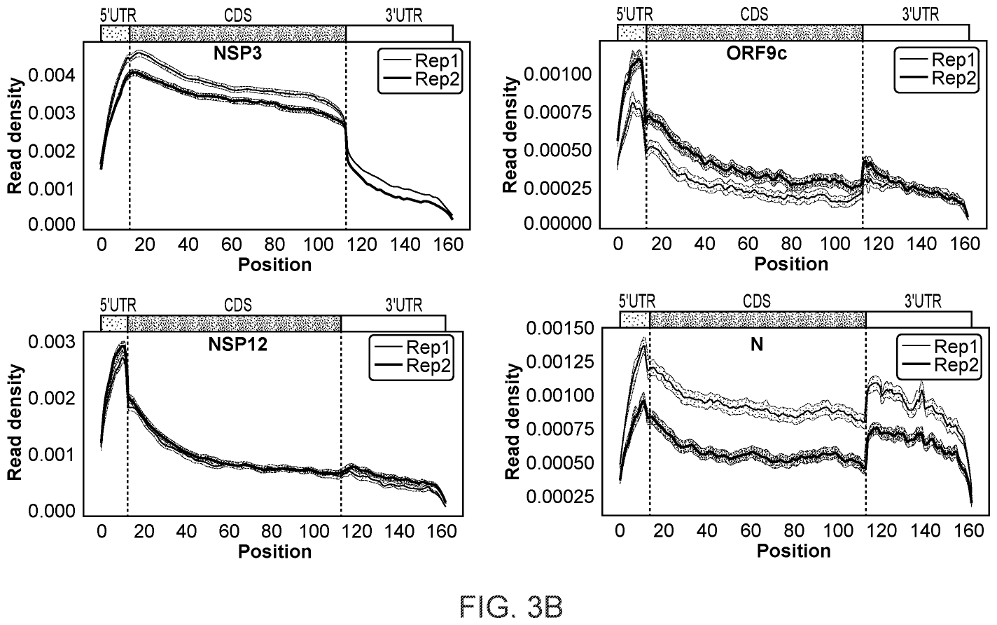

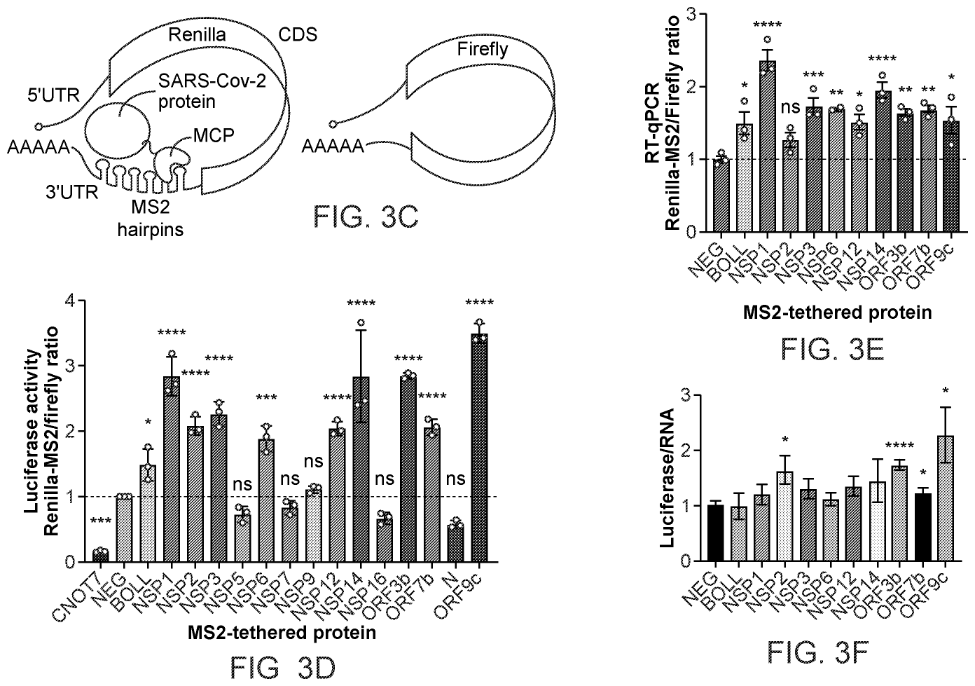

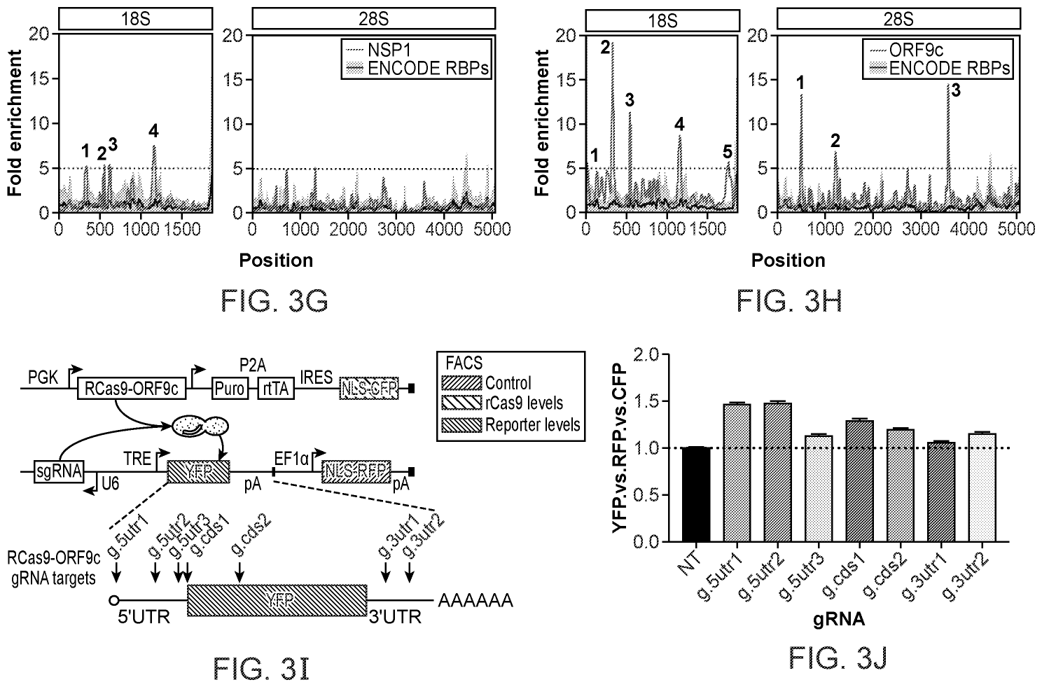

By examining the regional binding preferences of each SARS-CoV-2 protein, it was found that SARS-CoV-2 proteins are enriched at distinct regions of target mRNAs, which imply different regulatory functions because of the protein-RNA interaction. Aggregating the analysis of all targeted peaks for each SARS-CoV-2 protein identifies RNA regions that are preferentially bound (FIG. 3a). Of note, NSP12, ORF3b, ORF7b and ORF9c show the highest proportion of peaks in the 5′ UTR, NSP2, NSP3, NSP6 and NSP14 show the highest proportion of peaks in the coding region (CDS), NSP5, NSP7 and NSP9 display a high proportion of peaks in intronic regions, and N and NSP15 show the largest proportion of peaks in the 3′ UTR. Afiner-grained metagene analysis of read density across all target mRNA transcripts was also performed, where each of the 5′ UTR, CDS and 3′ UTR regions in an mRNA are scaled to standardized lengths (FIG. 3b). It was found that even though NSP2 has a similar number and proportion of peaks in the CDS as NSP3, it mainly targets the region spanning the 5′ UTR and coding start. In contrast, NSP3 reads, along with that of NSP6 and NSP14, coat the entire CDS, with a slight bias towards the start of the coding sequence.

Since 8 of the SARS-CoV-2 proteins—NSP2, NSP3, NSP6, NSP12, NSP14, ORF3b, ORF7b and ORF9c—have binding preferences at the 5′ UTR and CDS, it was hypothesized that their protein-RNA interactions could affect expression of the target mRNAs at the level of RNA turnover or translation. To evaluate the functional role of the specific protein-RNA interactions of SARS-CoV-2 proteins and target transcripts, 14 of the proteins were characterized using the tethered function reporter assays (FIG. 3c). The individual proteins were fused with an MS2 phage coat protein (MCP), which localizes the tagged protein to MS2 aptamer hairpins inserted in the 3′ UTR of Renilla luciferase. A firefly luciferase without MS2 hairpins is included as a control for non-specific effects of the viral protein. Plasmids encoding the MCP-tagged proteins and reporter constructs are co-transfected into HEK293T cells. Changes in Renilla luciferase activity normalized to firefly luciferase activity measures up- or downregulation of protein expression via either translation or mRNA stability because of positioning the MCP tagged protein in the vicinity of the Renilla mRNA. The luciferase readout does not by itself distinguish between translational or mRNA stabilizing effects.

From the tethering experiments, it was found that the ratio of Renilla-MS2 to firefly luciferase for 9 of the 14 SARS-CoV-2 proteins increase 1.9 (NSP6) to 3.5-fold (ORF9c) relative to FLAG-MCP control (p-value <0.002, two tailed multiple t-test) (FIG. 3d). Interestingly, these SARS-CoV-2 proteins display a stronger effect on mRNA translation than the tethering of BOLL (1.5-fold), which is a human RBP previously characterized to be amongst the strongest upregulators from a screen of more than 700 human RBPs. Even though NSP1 was found to bind to very few host mRNAs and its peaks are not mapped to the 5′ UTR and CDS, the results for NSP1 are consistent with its ability to enhance the transcription and translation of its own mRNA via interacting with the 5′ UTR of the genomic viral mRNA. Of the remaining 5 SARS-CoV-2 proteins, only NSP5, NSP16 and N display slight (but not significant) down-regulation effects (0.73-fold to 0.58-fold) compared to the FLAG peptide control, but to a lesser extent than that of the known translation repressor CNOT7 (0.16-fold). NSP7 and NSP9 appear to have no effect on the targeted expression of the Renilla reporter. To understand if the upregulation is occurring at the RNA or protein level, RT-qPCR was performed to measure the ratio of Renilla-MS2 to Firefly mRNAs. For all the enhancing proteins except for NSP2, the Renilla-MS2/Firefly mRNA ratio is significantly increased (p<0.05) compared to wildtype, albeit to different extents for different proteins (FIG. 3e). Of note, ORF9c shows the greatest enhancing effect (3.5-fold) in the dual luciferase assay, but its effect on the reporter RNAs is middling (1.5-fold). Taking the fold change in luciferase activity ratio to RNA ratio, ORF9c displays the greatest extent of upregulation at the protein level compared to RNA (2.3-fold) (FIG. 3f), followed by NSP2 and ORF3b (1.6 and 1.7 fold respectively). The rest of the proteins range from 1.1-fold (NSP6) to 1.5-fold (NSP14), compared to 1.0-fold of BOLL, suggesting that upregulation likely occurs at both the RNA and protein level.

To understand the origin of increase in mRNA translation, eCLIP reads were mapped to the 18S and 28S ribosomal subunits to determine if there are any specific interactions with the ribosome. Fold enrichment was determined directly from comparing read coverage in IP to size-matched input. It was found that enrichment peaks (>5-fold) of NSP1 reads are mostly mapped to the mRNA entry channel of 40S ribosome corresponding to helix 16 (peak2) and 18 (peak 3) of 18S rRNA, which is consistent with several cryo-EM structure data showing that NSP1 blocks the mRNA entry channel to inhibit host translation (FIG. 3g). In addition, a NSP1-binding peak was also observed mapped to helix 26/26a (peak 4) of 18S rRNA, a location important for hepatitis C viral internal ribosome entry site (IRES) element binding to the ribosome. This provides further evidence that the function of NSP1 is not only to block the host translation, but that it also may be involved in the regulation of viral RNA translation through mediating the interaction of SARS-CoV-2 5′ UTR/IRES with the ribosome. The impact of NSP1 enrichment at helix 10 (peak 1), an exposed flexible region of 18S rRNA, is unclear.

Unlike NSP1, ORF9c shows enrichment at both 28S and 18S rRNA. One of the major enriched regions of ORF9c on 28S rRNA is above the surface of 60S ribosome. This region consists of two ORF9c binding peaks (28S peak 1 and 2) that correspond to two helices, which are connected by their interactions with RPL4 and interact with RPL27a and RPL7 respectively. RPL4 has been shown to interact with RPL7 and further protrude into the core of 60S ribosome and associate with the peptide exit tunnel. The other major region of ORF9c binding to the ribosome is at the intersubunit interface which comprises a helix H63/ES27 (28S peak 3) of 28S rRNA, and two helices, helix 10 (18S peak 2) and 44 (18S peak 5), of 18S rRNA. These helices interact with RPL19, RPL24, RPS6, and RPS8, and have been shown to contribute to establishing eukaryote-specific intersubunit bridges. The interactions of ORF9c at the above two regions suggest that ORF9c may play a role in joining two ribosomal subunits to optimize ribosome function. The last ORF9c binding region is around the mRNA entry channel of 18S rRNA corresponding to helix 16 (18S peak 3), and two nearby helices, helix 1 (18S peak 1), and helix 26/26a (18S (peak 4)). Due to the relatively small size of ORF9c, its binding at helix 16 suggests it may play a role in regulating translation initiation by altering the position of helix 16. The metagene density plot for ORF9c shows binding mainly in the 5′ UTR of target mRNAs. By stabilizing the ribosomal complex, ORF9c may enhance translation efficiency of its target mRNAs at the start of translation. In addition, the binding of ORF9c at helix 1 and 26/26a implies it may mediate the interaction of SARS CoV2 5′UTR/IRES to host ribosome. Taken together, the results indicate ORF9c may be involved in optimizing ribosome structure and regulating translation initiation.

As an orthogonal validation and further evaluation of whether there is any regional effect in binding and upregulation of protein expression, ORF9c was fused to RNA-targeting Cas9 (RCas9) and its effect on mRNA translation of a reporter substrate was assessed. It was previously shown that regional binding preferences were not captured by the MS2-tethering assay, as human RBPs that bind to all three regions were found to regulate the expression of the targeted reporter, which was brought into proximity. Using 7 guide RNAs that tiled across the mRNA encoding yellow fluorescent protein (YFP) (Table 1), it was found that RCas9-ORF9c fusions upregulated the expression of YFP mRNA when targeted to its 5′ UTR. This regional preference is supported by the metagene read density analysis as well (FIG. 3b). Since most translational regulation occurs at the translation initiation step where the translational machinery assembles at the 5′ UTR, ORF9c targeting 5′ UTR of mRNAs suggests a potential role in upregulating the protein expression of target transcripts.

Taken together, these results suggest that SARS-CoV-2 proteins with a preference for binding to 5′ UTR and CDS regions have a capacity for upregulating the expression of target mRNAs. The increase in ultimate translation output was due to effects at both the RNA stabilization level and the translation enhancing level. Mapping eCLIP reads of ORF9c to 18S and 28S rRNA implies a role in enhancing translation and redirecting translation to target mRNAs.

TABLE 1

| | Sequence 5′ → 3′ | SEQ ID |
|---|---|---|
| gRNA Name | (mRNA sequence) | NO |
| g.5utr1 | ttttgacctccatagaagac | 1 |
| g.5utr2 | ggacggacgccagcgctaag | 2 |
| g.5utr3 | atccccgggtaccggtcgcc | 3 |
| g.cds1 | cggtcgccaccatggtgagc | 4 |
| g.cds2 | gaccaccttcggctacggcc | 5 |
| g.3utr1 | attcttacgctgagtacttc | 6 |
| g.3utr2 | catggcattccacttatcac | 7 |

### Example 4—NSP12 Upregulates Genes in Mitochondria and N-Linked Glycosylation Processes

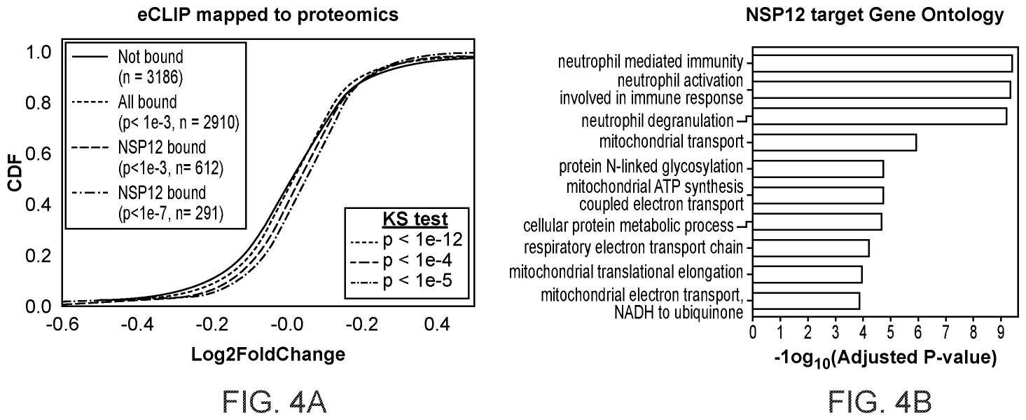

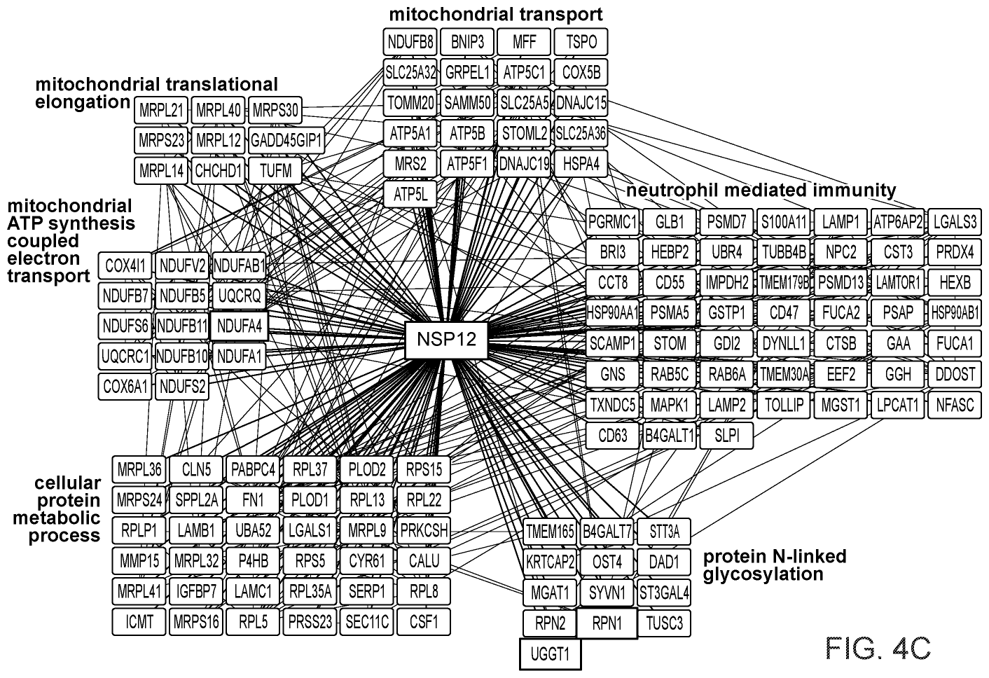

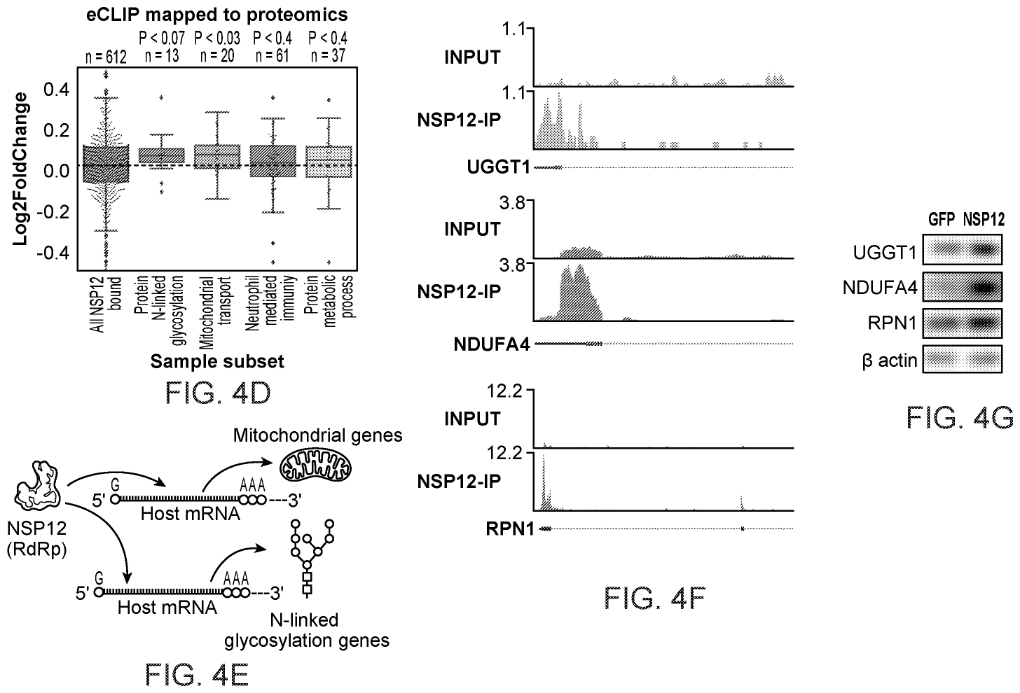

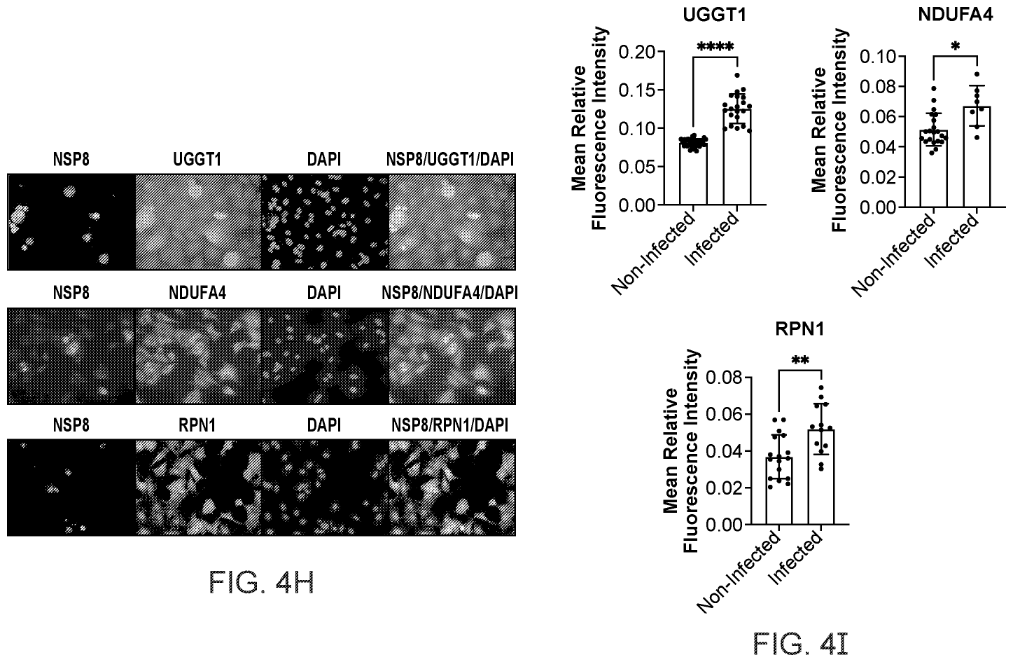

Based on the results of the two reporter assays, it was conjectured that SARS-CoV-2 proteins that bind to the 5′ UTR and CDS of its target genes upregulate gene expression. eCLIP target genes were mapped to existing proteomics datasets from SARS-CoV-2 infected cells and it was found that of the differentially expressed proteins (p<0.05, 24 hours post infection), proteins that are eCLIP targets with IDR reproducible peaks are expressed at higher levels than the non-targeted genes (p<10⁻¹² by Kolmogorov-Smirnoff (KS) test) (FIG. 4a). NSP12 targeted genes also appear to be less downregulated (p<10⁻⁴, KS test) due to SARS-Cov2 infection, with genes bound by more significant peaks showing a greater difference (p<10⁻⁵) (FIG. 4a). However, the opposite is observed in transcriptomics data from SARS-CoV-2 infected cells. eCLIP target genes show decreased RNA abundance (p<10⁻⁸), with NSP12 targeted genes appearing even more downregulated (p<10⁻²⁷). This may need to be understood in the complex context of regulation and counter regulation in viral-host relationships. There may be certain processes that are downregulated due to global transcription shutdown, but post transcriptional upregulation as exerted by NSP12 may upregulate specific genes to the advantage of the virus.

The GO processes enriched by the genes targeted by NSP12 include those related to neutrophil mediated immunity, mitochondrial processes (transport, translation elongation, ATP synthesis coupled electron transport), protein N-linked glycosylation and other cellular protein metabolic process (FIG. 4c, d). Among these processes, NSP12 targeted mitochondrial transport genes are the most significantly upregulated (p<0.03, KS test) compared to non-eCLIP target genes (FIG. 4e). To confirm whether individual genes in these pathways are upregulated by NSP12, genes from the top GO terms that are targeted by NSP12 in the 5′UTR region were selected, which are representative of the metagene profile for NSP12 (FIG. 3b, FIG. 3f). Among the N-linked glycosylated GO term genes, Ribophorin I (RPN1) is part of an N-oligosaccharyl transferase complex that links high mannose oligosaccharides to asparagine residues found in the Asn-X-Ser/Thr consensus motif of nascent polypeptide chains, and UDP-Glucose Glycoprotein Glucosyltransferase 1 (UGGT1) is a soluble protein of the endoplasmic reticulum (ER) that selectively reglucosylates unfolded glycoproteins. Represented in the mitochondrial ATP synthesis coupled electron transport and the respiratory electron transport chain GO processes, NDUFA4 is part of the enzyme cytochrome-c oxidase (or complex IV) and is important for its activity and biogenesis. NSP12 was exogenously introduced by transiently transfecting HEK293T cells, and by comparing to a control where a GFP plasmid was transfected, it was found by Western blotting that UGGT1, RPN1 and NDUFA4 are expressed at higher levels (FIG. 4g). In a human lung carcinoma cell line clonally overexpressing ACE2 (A549-ACE2), it was found by immunofluorescence that all three proteins appear induced in SARS-CoV-2 infected cells (stained for NSP8) (FIG. 4h, i), confirming the relevance of these induced genes to the actual viral infection.

Here, it was demonstrated that overexpression of NSP12, as well as SARS-CoV-2 virus infection in cells, enhances the expression of N-linked glycosylation related genes, UGGT1 and RPN1, and the mitochondrial cytochrome c oxidase subunit NDUFA4. Since N-linked glycosylation of host ACE2 receptor and virus Spike protein are important for their interactions and virus entry, the results suggest that the SARS-CoV-2 infection could activate the N-linked glycosylation pathway to facilitate the viral-host interaction and virus entry through NSP12. Upregulation of NDUFA4 by NSP12 may also imply a role in modulating mitochondrial bioenergetics during virus infection, as viral biogenesis depends on energy and metabolic resources provided by the host.

### Example 5—NSP9 Associates with the Nuclear Pore to Block mRNA Export

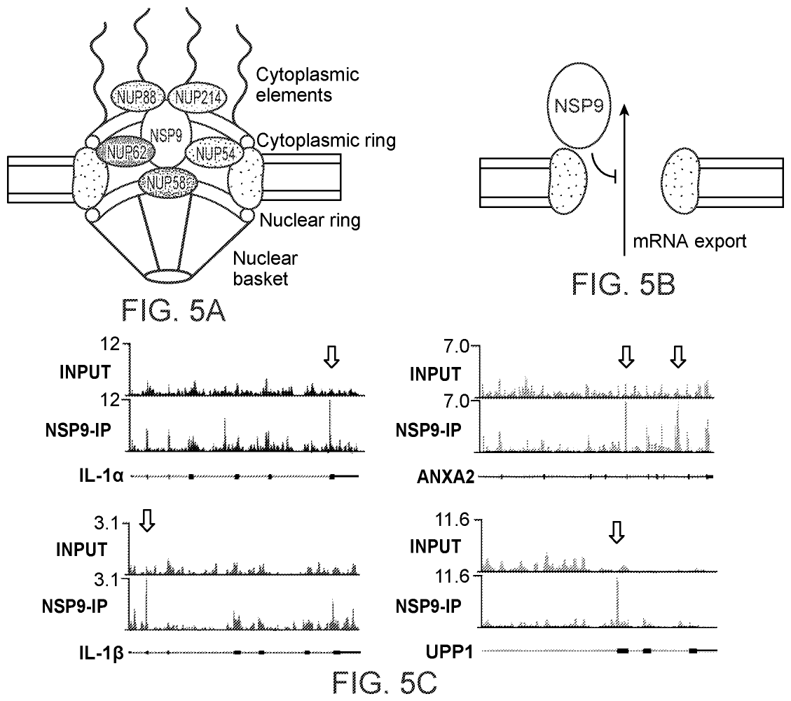

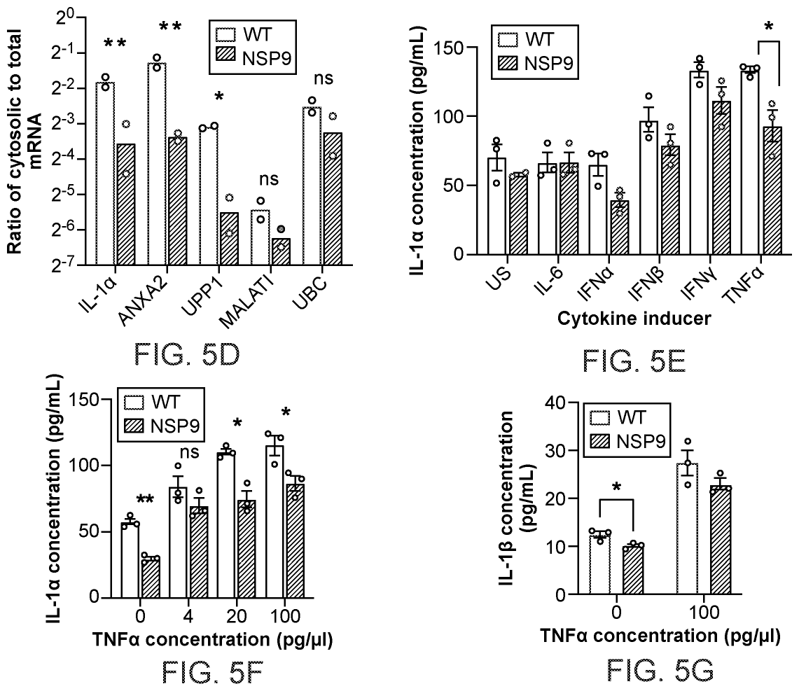

Using affinity mass-spectrometry, it was shown that NSP9 interacts with several nuclear pore complex proteins, including NUP62, NUP214, NUP88, NUP54 and 396 NUP581 (FIG. 5a). It was confirmed that NUP62 indeed co-immunoprecipitated with NSP9, which led to the hypothesis that NSP9 may interfere with mRNA export by associating with the nuclear pore (FIG. 5b). To determine if NSP9 inhibits mRNA export activity, the mRNA levels of NSP9 target genes in cytoplasmic and nuclear fractions were assayed. Both NSP9 expressing BEAS-2B cells and the parental or wild type BEAS-2B cells were fractionated into nuclear and cytoplasmic fractions, followed by RNA extraction and RT-qPCR of target genes. NSP9 target genes were observed to have significant peaks near the 3′ splice site, which may suggest interference of splicing-coupled export (FIG. 5c). It was found that target genes IL-1α, ANXA2 and UPP1 had lower cytosolic to total mRNA ratios in NSP9-expressing versus parental cells, whereas the cytosolic mRNA levels of non-targeted control genes MALAT1 and UBC were not significantly lowered (FIG. 5d). Even though nuclear RNA fractions were purified at high yields (>1 μg/μl), the RT-qPCR CT values of the target genes were too high (>25 cycles) for accurate quantification. Interleukin 1α (IL-1α) is an important inflammatory cytokine constitutively produced in epithelial cells and plays a central role in regulating immune responses, including being a master cytokine in acute lung inflammation induced by silica micro- and nanoparticles. Interleukin 1β (IL-1β) binds to the same IL1 receptor as IL-1α, and its mRNA is bound by NSP9 even though it does not pass the IDR threshold. To determine if NSP9 inhibiting the nucleocytoplasmic export of the mRNA of IL-1α has any impact on the production of this cytokine, an ELISA was performed on the growth media of BEAS-2B wild type and NSP9 expressing cells 48 hours after induction by several common cytokines. Interferon α, β and γ resulted in lowered IL-1α levels in NSP9 cells compared to wild type, though tumor necrosis factor alpha (TNFα) resulted in the greatest reduction (˜30%) (FIG. 5e). The observation of reduced IL-1α produced at different concentrations of TNFα (FIG. 5f) was reproduced. In addition, it was observed that reduced IL-13 was produced in NSP9 expressing cells than in wildtype BEAS-2B cells (FIG. 5g). Thus, NSP9 association with the nuclear pore complex proteins aligns with the observation of decreased cytoplasmic abundance of NSP9 target mRNAs, suggesting that NSP9 interaction may directly inhibit nuclear export. Further, NSP9 reduced the production of its target gene IL-1α, which suggests that the export inhibition mechanism may be a strategy that SARS-CoV-2 employs to dampen inflammatory host response.

### Example 6—SARS-CoV-2 Protein-Host RNA Interactions Identify Potential Therapeutic Targets

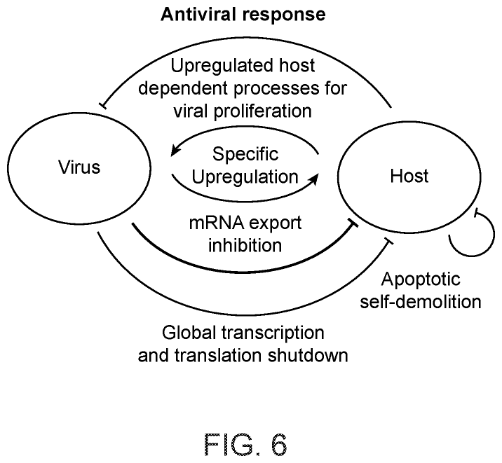

Like many viruses, the host-viral interactions underlying SARS-CoV-2 infection is broadly understood in terms of the virus hijacking the host cell by globally shutting down the expression of host genes that are irrelevant or hostile to its replication, while the host attempts to fight off the virus by mounting apoptotic and inflammatory responses. To add to this understanding, it was proposed that viral proteins interact with host RNAs to activate a subset of host genes for its own survival through targeted translation activation or mRNA stabilization (FIG. 6). It was shown that NSP12 specifically upregulates genes in the processes of protein N-linked glycosylation and mitochondrial ATP synthesis and transport. While it has been shown that NSP1 is a global repressor of host cell transcription and translation, it was also proposed that NSP9 contributes another layer to dampening host gene expression by inhibiting mRNA export. Understanding specifically upregulated processes and genes will enable the development of new antiviral strategies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNA recognition complex comprising:
(a) an RNA-targeting agent; and
(b) a coronavirus-derived protein, comprising a NSP1, a NSP2, a NSP3, a NSP6, a NSP12, a NSP14, a ORF3b, a ORF7b, or a ORF9c protein.

2. The RNA recognition complex of claim 1, further comprising a linker.

3. (canceled)

4. The RNA recognition complex of claim 1, wherein the RNA-targeting agent comprises an RNA-targeting Cas effector.

5. (canceled)

6. (canceled)

7. (canceled)

8. (canceled)

9. (canceled)

10. (canceled)

11. The RNA recognition complex of claim 1, wherein the RNA-targeting agent further comprises a single guide RNA (sgRNA), wherein the sgRNA is targeted to an individual gene of a cell.

12. The RNA recognition complex of claim 11, wherein the sgRNA is selected from a group consisting of SEQ ID NOs: 1-7.

13. (canceled)

14. (canceled)

15. (canceled)

16. A method of modulating gene expression of a target RNA comprising:
delivering a RNA recognition complex into a cell, wherein the RNA recognition complex comprises a RNA-targeting agent, and a SARS-CoV-2 coronavirus-derived protein, and wherein the RNA recognition complex binds to the target RNA and modulates gene expression of the target RNA in the cell.

17. The method of claim 16, wherein the method further comprises profiling the gene expression of the target RNA in the cell, wherein the gene expression is upregulated.

18. (canceled)

19. The method of claim 16, wherein the coronavirus-derived protein comprises a NSP1, a NSP2, a NSP3, a NSP6, a NSP12, a NSP14, a ORF3b, a ORF7b, or a ORF9c protein.

20. The method of claim 16, wherein the method further comprises profiling the gene expression of the target RNA in the cell, wherein the gene expression is downregulated.

21. The method of claim 20, wherein the coronavirus-derived protein comprises a NSP9 protein.

22. The method of claim 17, wherein the profiling comprises transcriptome analysis or gene expression analysis.

23. The method of claim 17, wherein the profiling comprises enhanced cross-linking immunoprecipitation (eCLIP).

24. (canceled)

25. The method of claim 16, wherein the RNA-targeting agent comprises an RNA-targeting Cas effector.

26. (canceled)

27. (canceled)

28. (canceled)

29. (canceled)

30. (canceled)

31. (canceled)

32. The method of claim 16, wherein the RNA-targeting agent further comprises a single guide RNA (sgRNA), wherein the sgRNA is targeted to the target RNA in the cell.

33. The method of claim 32, wherein the sgRNA is selected from a group consisting of SEQ ID NOs: 1-7.

34. A method of treating a disease associated with reduced gene expression in a subject in need thereof, the method comprising:
administering a RNA recognition complex to the subject, wherein the RNA recognition complex comprises a RNA-targeting agent, and a SARS-CoV-2 coronavirus-derived protein, and wherein the RNA recognition complex binds to the target RNA and upregulates gene expression of the target RNA in the cell, thereby treating the disease associated with reduced gene expression.

35. (canceled)

36. The method of claim 34, wherein the RNA-targeting agent comprises an RNA-targeting Cas effector.

37. (canceled)

38. (canceled)

39. (canceled)

40. (canceled)

41. (canceled)

42. (canceled)

43. The method of claim 36, wherein the RNA-targeting agent further comprises a single guide RNA (sgRNA), wherein the sgRNA is targeted to the target RNA in the cell.

44. The method of claim 43, wherein the sgRNA is selected from a group consisting of SEQ ID NOs: 1-7.

45. (canceled)

46. The method of claim 34, wherein the coronavirus-derived protein comprises a NSP1, a NSP2, a NSP3, a NSP6, a NSP12, a NSP14, a ORF3b, a ORF7b, or a ORF9c protein.

47. (canceled)

48. (canceled)

49. (canceled)


SEQUENCE LISTING (7 sequences)

SEQ ID NO: 1 - DNA, 20 bp, Artificial
  gRNA - g.5utr1
ttttgacctccatagaagac

SEQ ID NO: 2 - DNA, 20 bp, Artificial
  gRNA - g.5utr2
ggacggacgccagcgctaag

SEQ ID NO: 3 - DNA, 20 bp, Artificial
  gRNA - g.5utr3
atccccgggtaccggtcgcc

SEQ ID NO: 4 - DNA, 20 bp, Artificial
  gRNA - g.cds1
cggtcgccaccatggtgagc

SEQ ID NO: 5 - DNA, 20 bp, Artificial
  gRNA - g.cds2
gaccaccttcggctacggcc

SEQ ID NO: 6 - DNA, 20 bp, Artificial
  gRNA - g.3utr1
attcttacgctgagtacttc

SEQ ID NO: 7 - DNA, 20 bp, Artificial
  gRNA - g.3utr2
catggcattccacttatcac